(19) United States
(12) Patent Application Publication
(10) Pub. No.: US 2023/0202956 A1
(43) Pub. Date: Jun. 29, 2023

(54) PROCESS FOR ACETIC ACID PRODUCTION BY REMOVING PERMANGANATE REDUCING COMPOUNDS

(71) Applicant: Celanese International Corporation, Irving, TX (US)

(72) Inventors: David LEE, Houston, TX (US); Yaw-Hwa LIU, Missouri City, TX (US); Ronald D. SHAVER, Houston, TX (US)

(73) Assignee: Celanese International Corporation, Irving, TX (US)

(21) Appl. No.: 17/928,565

(22) PCT Filed: Jun. 3, 2021
(86) PCT No.: PCT/US2021/035687

§ 371 Date: Nov. 29, 2022

Related U.S. Application Data
(60) Provisional application No. 63/034,072, filed on Jun. 3, 2020.

(51) Int. Cl. C07C 51/12; C07C 51/44

(52) U.S. Cl. CPC C07C 51/12; C07C 51/445

(57) ABSTRACT

This process relates to controlling acetal formation when removing acetaldehyde from a methanol carbonylation process using an extractive distillation column. Acetals may be formed by a secondary reaction of acetaldehyde and an alcohol (such as methanol). The process controls the formations to prevent excess acetal accumulation in the lower stream from the extractive distillation column.

### PRIORITY

This application claims priority to U.S. Provisional Application No. 63/034,072, filed on Jun. 3, 2020, which is incorporated herein by reference for all purposes.

### FIELD OF THE INVENTION

This invention relates generally to a process for production of acetic acid and to improved processes for controlling or maintaining the total amount of acetal, on a mass basis, in a lower stream during distilling, including extractive distillation, to remove or reduce acetaldehyde and other permanganate reducing compounds (PRCs).

### BACKGROUND OF THE INVENTION

Methanol carbonylation is an industrial process for obtaining acetic acid in the presence of a reaction mixture containing homogeneous catalyst. Despite the high yields of acetic acid, the process is known to generate impurities resulting in low purity acetic acid. One such impurity that has received considerable attention is acetaldehyde because of the relevant difficulty in removal, acetaldehyde is a precursor to several other impurities, and the impact on purity of acetic acid. For example acetaldehyde has a close boiling point to an effective catalyst promoter, which makes simple distillation insufficient. To overcome these insufficiencies, several proposals have been used to remove acetaldehyde by alkane or water extraction, or by reaction with amino compounds, oxygen-containing gases, and hydroxyl compounds. Unfortunately, despite the use of these treatments, acetaldehyde continues to be challenge in obtaining high purity acetic acid. Further, formation of acetaldehyde derived impurities reduces the efficiency when removing acetaldehyde.

Additional designs for distilling and/or extracting have been proposed to remove acetaldehyde, but continue improvements are needed to achieve a high quality acetic acid product.

U.S. Pat. No. 8,859,810 describes processes for producing acetic acid and, in particular, to improved processes for recovering permanganate reducing compounds formed during the carbonylation of methanol in the presence of a carbonylation catalyst to produce acetic acid. Alkyl halides are removed or reduced from the recovered permanganate reducing compounds.

U.S. Pat. No. 10,562,836 describes a process for producing acetic acid while efficiently separating permanganate reducing compounds (PRCs) and methyl iodide. PRCs are separated or removed from a mixed composition containing PRCs and methyl iodide by distilling the mixed composition in a distillation step to form an overhead stream, a side-cut stream, and a lower stream. In a distillation column of the distillation step, an extractant (e.g., water) extracting PRC's preferentially to methyl iodide is added to a concentration zone in which PRC's and methyl iodide are concentrated, and an extraction mixture falling from the concentration zone is withdrawn as the side-cut stream.

U.S. Pat. No. 10,265,639 describes a process for separating or removing PRCs from a first mixture containing at least one PRC, methyl iodide, and water comprises the steps of: feeding the first mixture to a feed port of a distillation column, and distilling and separating the first mixture into an upper stream and a lower stream, wherein the distillation of the first mixture forms a second mixture at an upper position than the feed port, and the process further comprises the steps of: withdrawing the second mixture as the upper stream, and withdrawing the lower stream from a lower position than the feed port.

Although existing carbonylation processes are highly efficient, further improvements for the recovery of acetic acid while reduction in impurities in a safe and efficient manner continue to be desirable.

### SUMMARY OF THE INVENTION

This invention generally relates to processes for the production of acetic acid. The embodiments of the present invention provide processes for effectively removing permanganate reducing compounds (PRCs) from one or more C₁-C₁₂ alkyl iodides (in particular methyl iodide) while reducing the total acetal mass composition in a return stream and for producing acetic acid.

In one embodiment, there is provided a process for separating at least one permanganate reducing compound (PRC; such as acetaldehyde) from a first mixture comprising the at least one PRC, one or more C₁-C₁₂ alkyl iodides (such as methyl iodide) and water, the process comprising the steps of feeding the first mixture to a first location of a distillation column, wherein the first mixture has a methanol mass composition that is less than the difference, in terms of mass, between the one or more C₁-C₁₂ alkyl iodides and water, distilling the first mixture into a second mixture and a lower stream, the second mixture being at least one stream selected from the group consisting of an overhead stream and a sidedraw stream being withdrawn higher than that of the first location, and withdrawing the lower stream from a second location lower than the first location, and wherein the lower stream satisfies at least one (or two) of the following conditions (i) to (iii):
(i) a total acetal mass composition in the lower stream is not more than 0.02 wt %;
(ii) a total acetal mass composition in the lower stream is less than the combined mass composition of methanol and acetaldehyde in the lower stream; or
(iii) a total acetal mass composition in the lower stream is less than the methanol mass composition in the first mixture;
   In one embodiment, according to this process the first mixture is separated into the second mixture and lower stream without the supply of additional methanol. Further the resulting second mixture is enriched in the at least one PRC.

In one embodiment, there is provided an acetic acid production process that comprises the following steps for controlling the formation of acetals allowing methanol to continuously react with carbon monoxide in the presence of a reactive mixture (comprising a metal catalyst, an ionic metal iodide, and methyl iodide, acetic acid, methyl acetate, and water), evaporating the reaction mixture (with or without heating) to yield a vapor crude product and a catalyst recycle stream, distilling at least a portion of the vapor crude product to form an overhead and a side stream and condensing the overhead in one or more condensers and collecting the condensate(s) in a receiver into an upper phase and a lower phase, separating at least a portion of the lower phase (and/or at least a portion of the upper phase) to form a second stream comprising acetaldehyde and a lower stream withdrawing the lower stream from a second location lower than the first location. The lower stream may satisfy at least one (or two) of the following conditions (i) to (iii):
(i) a total acetal mass composition in the lower stream is not more than 0.02 wt %;
(ii) a total acetal mass composition in the lower stream is less than the combined mass composition of methanol and acetaldehyde in the lower stream; or
(iii) a total acetal mass composition in the lower stream is less than the methanol mass composition in the lower phase;
   In the embodiments for separating the lower phase (and/or upper phase) into the second mixture and lower stream without the supply of additional methanol. In addition, the second mixture is enriched in the at least one PRC over the lower phase (and/or upper phase).

### BRIEF DESCRIPTION OF THE DRAWINGS

The present disclosure will be better understood in view of the appended non-limiting figures, wherein:

FIG. 1 illustrates a schematic process for producing acetic acid in accordance with embodiments of the present invention.

FIG. 2 illustrates a schematic process for separating and removing acetaldehyde in accordance with embodiments of the present invention.

FIG. 3 illustrates another schematic process for separating and removing acetaldehyde in accordance with embodiments of the present invention.

At the outset, it should be noted that in the development of any such actual embodiment, numerous implementation-specific decisions must be made to achieve the developer's specific goals, such as compliance with system-related and business-related constraints, which will vary from one implementation to another. In addition, the processes disclosed herein can also comprise components other than those cited or specifically referred to, as is apparent to one having average or reasonable skill in the art.

As is evident from the figures and text presented herein, a variety of embodiments are contemplated.

### DETAILED DESCRIPTION OF THE INVENTION

The embodiments of the present invention provide processes for effectively removing permanganate reducing compounds (PRCs), which can otherwise deteriorate the quality of the acetic acid, from one or more C₁-C₁₂ alkyl iodides while reducing the total acetal mass composition in a return stream. High amounts of total acetal reduce the ability to remove PRCs, resulting in ineffective processing. In one embodiment, to reduce the total acetal, the feed stream to the distillation column has a methanol mass composition that is maintained (controlled) to be less than the difference based on mass between the one or more C₁-C₁₂ alkyl iodides (in particular methyl iodide) and water in the stream. Reducing the total amount of acetal (and/or hemiacetals) may further enrich the mixture in PRC. As used herein the terms “mass composition” or “concentration” refers to the mass fraction of substance to the total mass and is generally expressed in % by mass or wt %, unless indicated otherwise.

In another embodiment, to reduce the total acetal the process may involve separating PRCs from the one or more C₁-C₁₂ alkyl iodides without an additional supply of methanol to the distillation column.

In another embodiment, to reduce the total acetal the process may involve separating PRCs from the one or more C₁-C₁₂ alkyl iodides in the presence of acetic acid, water, methyl acetate and/or methanol.

The present inventors have found that acetals formation proceeds to react one of the PRCs to form high boiling point components that tend to concentrate in (transfer to) the lower stream. During methanol carbonylation, acetaldehyde or byproducts of acetaldehyde present in the mixture to the distillation column may undergo acetalization to produce acetals and/or hemiacetals. The acetalization of acetaldehyde (AcH) to acetal (e.g., 1,1-dimethoxyethane) is a two-step acid catalyzed reaction in methanol carbonylation system. The first step the acetaldehyde reacts with methanol to form the hemiacetal. The second step the hemiacetal reacts with methanol to yield the acetal, 1,1-dimethoxyethane, and water. The overall reaction is shown in Formula I as:

CH₃CHO+2 CH₃OH⇄(CH₃O)₂CHCH₃+H₂O Formula 1

The acetalization of acetaldehyde (AcH) to acetal (e.g., 1,1-dimethoxyethane) is an equilibrium (reversible) reaction as shown:

$$Keq = \frac{[ Acetal ] [ {H}_{2} O ]}{{[ AcH ] [ {CH}_{3} OH ]}^{2}}$$

The lower stream is a return stream, which refers to a stream that is returned to the process, such as returning to the reactor or an upstream distillation column or other suitable vessel. This undesirably introduces the acetal and/or hemiacetal throughout the process leading to a buildup of impurities that can deteriorate the quality of the acetic acid product.

In one embodiment, the process removes the PRC and withdraws a lower stream that satisfies at least one the following conditions (i) to (iii):
(i) A total acetal amount in the lower stream is not more than 0.02 wt %, e.g., not more than 0.018 wt %, not more than 0.015 wt % or not more than 0.01 wt %. In one embodiment, to remove acetals formed in the column, the lower stream may have a lower amount that is not less than 0.0001 wt %, e.g., not less than 0.0005 wt % or not less than 0.001 wt %.
(ii) A total acetal amount in the lower stream is less than the combined amount of methanol and acetaldehyde in the lower stream. In one embodiment, to effectively remove the PRC, the lower stream contains lower amounts of methanol and acetaldehyde on a mass basis. When these combined amounts are high, the total acetal mass composition may also be relatively large. This reduces the efficiency in removing PRC due to the buildup in the lower stream. The methanol and acetaldehyde mass compositions are independent and in some embodiments there may be absence methanol or acetaldehyde. In one embodiment, the mass composition of methanol may be not more than 1.9 wt %, e.g., not more than 1.5 wt %, not more than 1 wt %, not more than 0.8 wt %, not more than 0.7 wt %, not more than 0.5 wt %, not more than 0.4 wt %, not more than 0.3 wt %. In one embodiment, the mass composition of acetaldehyde may be not more than 1.9 wt %, e.g., not more than 1 wt %, not more than 0.5 wt %, not more than 0.1 wt %, not more than 0.01 wt %, not more than 0.008 wt %, not more than 0.006 wt %. In one embodiment, the combined mass composition of methanol and acetaldehyde may be not more than 2 wt %, e.g., not more than 1 wt %, not more than 0.9 wt %, not more than 0.8 wt %, not more than 0.7 wt %, not more than 0.6 wt %, not more than 0.5 wt %, or not more than 0.1 wt %.
(iii) A total acetal mass composition in the lower stream is less than the methanol mass composition in the first mixture. Maintaining low mass compositions of methanol in the first mixture has been found to limit or reduce the total acetal mass compositions in the lower stream. In one embodiment, the mass composition of methanol in the first mixture may be not more than 2 wt %, e.g., not more than 1.5 wt %, not more than 1 wt %, or not more than 0.5 wt %.

In one embodiment, the lower stream satisfies at least two of the conditions (i) to (iii) described above to maintain a relatively low total acetal amount. This includes but is not limited to satisfying condition (i) and condition (ii), satisfying condition (i) and condition (iii), or satisfying condition (ii) and condition (iii). In a similar manner, there may be embodiments in which the lower streams satisfies each of the conditions (i), (ii) and (iii).

PRCs may include acetaldehyde, acetone, methyl ethyl ketone, butylaldehyde, crotonaldehyde, 2-ethyl crotonaldehyde, 2-ethyl butyraldehyde, and the aldol condensation products thereof. Even at low amounts these compounds can be evaluated by a permanganate reducing substance test (permanganate time) of the acetic acid product. A permanganate time for the acetic acid product may be not less than 70 minutes, e.g. not less than 90 minutes, not less than 110 minutes, not less than 120 minutes, not less than 150 minutes, not less than 250 minutes or not less than 300 minutes.

High amounts of total acetal reduce the ability to remove PRCs, resulting in ineffective processing. In one embodiment, the acetals may include but are not limited to 1,1-dimethoxyethane. In addition to acetals species, the total acetal amounts may also include hemiacetals thereof.

The lower stream may also contain other useful components, such as methyl iodide, methyl acetate, water, and/or acetic acid. Thus, it is desirable to return the lower stream in the process by directly or indirectly recycling to the reactor.

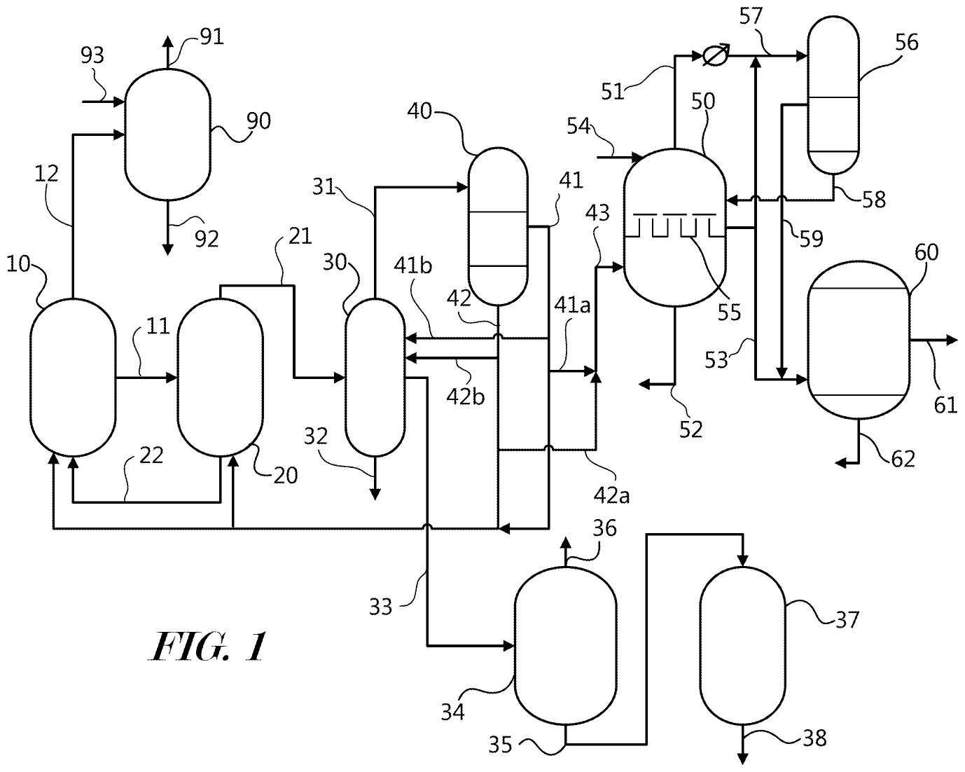

Hereinafter, one embodiment of the present invention will be described. In FIG. 1 there is shown a schematic continuous acetic acid process through carbonylation of methanol. This process comprises a reactor 10, an evaporator 20, distillation columns 30 and 34, resin vessel 37 such as an ion exchange resin, separation unit 40 such as a liquid-liquid separation unit, distillation column 50, vessel 60, and absorber 90. For purposes of the present disclosure, the lower stream 52 is obtained from the bottom or near the bottom of distillation column 50. In the embodiments described herein, these steps are not limited to these steps and the steps for producing acetic acid may exclude one or more of vessel 60, distillation column 34, resin vessel 37, and/or absorber 90. In some embodiments, there may be additional distillation column(s) (not shown) positioned upstream or downstream of resin vessel 37. Through this simple and efficient processing a product stream 38 may be obtained that comprises glacial acetic acid with low impurities. Additional distillation column(s) may be used for removing or separating other impurities as needed.

It should be understood to those skilled in the art that various processing equipment is now shown in detail in FIG. 1, including heat exchangers, receivers, pumps, controls, valves, etc. Unless stated otherwise, the absence of such processing equipment would be understood by an ordinary artisan that such processing equipment would be used as appropriate.

In one embodiment, the overhead 31 of column 30 may be condensed in one or more condensers and collected in separation unit 40. Separation unit 40 maintains conditions to form two phases, namely upper phase 41 comprised significantly of water and a lower phase 42 comprised significantly of C₁-C₁₂ alkyl iodides, including but not limited to methyl iodide. The upper phase 41 and/or lower phase 42 may contain PRCs and thus it is desirable to remove or reduce the amount of PRCs by introducing the upper phase 41a and/or lower phase 42a into the distillation column 50. The first mixture, which is introduced to the distillation column 50, may be either the upper phase 41a and/or lower phase 42a, and/or a combination of a portion these streams, through feed line 43. The first mixture may comprise at least one PRC, one or more C₁-C₁₂ alkyl iodides and water. To maintain lower total acetal amounts in the lower stream 52, the first mixture may have a relatively low amount of methanol. In one embodiment, the mass composition of methanol is less than the difference based on mass between the one or more C₁-C₁₂ alkyl iodides and water.

In one embodiment, the upper phase 41a and/or lower phase 42a (first mixture) obtained by the separation in distillation column 30 is introduced at a first location (mid-point between the column top and the column bottom) of a distillation column 50 through a feed line 43. In some embodiments, a solvent/extractant feed line 54 may be introduced above the feed line 43 so that extractive distillation is performed in the distillation column 50. The solvent may contain water, alkanes, or a combination thereof. The solvent may be supplied or introduced without any methanol or other alcohols. This allows the first mixture to be separated in distillation column 50 into the second mixture and lower stream without the supply of additional methanol. In other embodiments, solvent feed line 54 is absence.

The first mixture is distilled in the distillation column 50 under conditions sufficient to form a second mixture, which may be withdrawn as an overhead stream 51 or sidedraw 53, or a combination thereof. The second mixture is withdrawn at a location is that is higher, being closer to the column top, than that of the first location. In one embodiment, the second mixture may comprise a significant portion of the PRCs from the first mixture so that the PRCs are efficiently separated. In addition, to the second mixture, distillation column 50 also withdraws a lower stream 52. This lower stream 52 is withdrawn from a location that is lower, being closed to the column bottom, than that of the first location. The lower stream 52 may be withdrawn from the lower section, or near the base of the column. To satisfy the conditions for the embodiments disclosed herein, the lower stream 52 satisfies at least one of the following conditions (i) to (iii):
(i) a total acetal mass composition in the lower stream is not more than 0.02 wt %;
(ii) a total acetal mass composition in the lower stream is less than the combined mass composition of methanol and acetaldehyde in the lower stream; or
(iii) a total acetal mass composition in the lower stream is less than the methanol mass composition in the first mixture.

As described further herein, the PRCs may be further removed or separated from the second mixture. This reduces the total PRCs mass composition in the reactor and allows the production of a high quality acetic acid product.

Returning to reactor 10 in FIG. 1, this is a vessel for performing a reaction step. This reaction step continuously produces (step for continuously producing) acetic acid through a carbonylation reaction, preferably a methanol carbonylation reaction. The reaction step produces a reaction mixture 11, which may be continuously removed. Although, methanol and carbon monoxide are raw materials fed to the reactor 10, these raw materials are converted in the reactor and there are low unreacted mass compositions of these components in the reactor 10 as well as the reaction mixture 11. The reaction mixture also contains a metal catalyst, methyl iodide, methyl acetate, a co-catalyst, water, and acetic acid. In one embodiment, lower stream 52 may be recycled directly to reactor 10.

Evaporator 20 is a vessel for performing the flash distillation. This evaporator step removes (step for removing acetic acid) from the reaction mixture 11 in a vapor crude product 21 and returning the catalyst recycle stream (catalyst residuum stream) 22 to the reactor 10. In one embodiment, the lower stream 52 may be fed to evaporator 20 in the lower portion or may be combined with the catalyst recycle stream 22 and returned to the reactor 10. To stabilize the catalyst, and thus prevent undesirable precipitation, under the conditions in the evaporator 20, an usable stream of carbon monoxide may be introduced. In one embodiment, a portion of the vapor crude product 21 may be condensed and returned to reactor 10 to provide a temperature control.

Distillation column 30 is a vessel for separating the vapor crude product 21 from the evaporator 20. This distillation step is for further purifying the vapor crude product 21, and yields at least two of the following streams: an overhead 31, a bottoms 32, and an acetic acid stream 33. In some embodiments, the evaporator 20 and the distillation column 30 may be combined into a combined vessel (not shown) and the reactor mixture 11 is fed directly into this combined column. U.S. Pat. No. 7,790,920 describes such a combined column, and the entire contents and disclosures are incorporated by reference.

Overhead 31 may be condensed in an indirect condenser using a coolant, including but not limited to process water or cooling water, having a temperature from 20° C. to 120° C., e.g., from 20° C. to 90° C., from 25° C. to 80° C., or from 25° C. to 45° C., in one or more condensers. In one embodiment, the resultant condensate(s) are collected in separation unit (decanter) 40, which is maintained under conditions to cause the condensed overhead to phase separate. In one embodiment, separation unit 40 yields two phases, an upper phase 41 comprised significantly of water and a lower phase 42 comprised significantly of C₁-C₁₂ alkyl iodides, including but not limited to methyl iodide. Upper phase 41 and/or lower phase 42 may be refluxed to distillation column 30 and/or returned to reactor 10. For removal of PRCs, a portion of either the lower phase 42a and/or upper phase 41a are introduced into the second distillation column 50.

Acetic acid stream 33 comprises acetic acid, and has an increased mass composition of acetic acid than vapor crude product 21 by virtue of being enriched in distillation column 30. To further obtain a high quality product a portion of stream 33 may be fed to a purification step using distillation, absorption, extractive or other means to practically remove water, as well as other impurities, to obtain high quality acetic acid. High quality acetic acid may be referred to as glacial acetic acid and has very low water amounts, e.g., of less than or equal to 2000 ppm (weight ppm) or less than or equal to 1500 ppm. Other impurities, which include crotonaldehyde, butyl acetate, 2-ethyl-crotonaldehyde, propionic acid and formic acid, are similarly in very low amounts once removed.

Hereinafter, the embodiments will be explained in detail with reference to the figures as necessary. There is at least one corresponding unit for each step described in the various processes and may be referred to interchangeably.

Reactor (10) for Carbonylation Process

The reactor 10 is a unit for performing the carbonylation reaction step in the liquid phase using a homogenous catalyst. The reaction step is for carbonylating methanol to produce acetic acid in a continuous manner. The starting materials in the reaction are liquid methanol and gaseous carbon monoxide. A methanol-containing feed stream and carbon monoxide-containing feed stream are directed to reactor 10, in which the carbonylation reaction occurs to form acetic acid. Although not shown, a flow transmitter may be present on the both feed streams to control and/or monitor the flow of each respective stream. In particular, controlling and/or monitoring the mass flow of the methanol-containing feed stream is useful for determining the efficiency of the process.

Methanol-containing feed stream may comprise at least one member selected from the group consisting of methanol, dimethyl ether, and methyl acetate. For purposes of the process described herein commercially available methanol may be used. Various impurities, including aminic and/or metallic, may be removed or reduced from the methanol-containing feed stream in a pre-treatment step. Methanol-containing feed stream may be derived in part from a fresh feed from a reservoir tank (not shown), a recycled feed from the system, or a combination of fresh and recycle feeds. At least some of the methanol and/or reactive derivatives thereof will be converted to, and hence present as, methyl acetate in the liquid medium by esterification reaction with acetic acid. The methanol amounts in reactor 10, which refers to the unreacted methanol, may be less than or equal to 1 wt %, e.g., less than or equal to 0.8 wt %, less than 0.5 wt %, or less than 0.3 wt %.

Carbon monoxide-containing feed stream may comprise primarily carbon monoxide of greater than or equal to 95 vol. %, e.g., greater than or equal to 97 vol. % or greater than or equal to 99 vol. %. In some embodiments, minor impurities such as hydrogen, carbon dioxide, oxygen, and/or nitrogen may be present in amount of less than 5 vol. %, e.g., less than 3 vol. % or less than 1 vol. %. These minor impurities may also be generated by various side reactions under operating conditions.

The carbon monoxide partial pressure, at an absolute pressure, in the reactor may vary widely but is typically from 2 to 30 atm, e.g., from 3 to 18 atm or from 6 to 15 atm. The hydrogen, which may be generated in the reaction or may be supplied as needed, increases the catalytic activity but can also result in formation of byproducts, including acetaldehyde. The hydrogen partial pressure, at an absolute pressure, in the reactor is typically from 0.05 to 5 atm, e.g., from 0.25 to 2 atm or from 0.3 to 1.8 atm. Because of the partial pressure of byproducts, which is typically less than 1 atm, and the vapor pressure of the contained liquids, the total reactor internal pressure may range from 15 to 40 atm (absolute pressure), e.g., from 20 to 35 atm. In some embodiments, the internal pressure of the reactor may be controlled by withdrawing or venting a gaseous stream 12.

Typical carbonylation reaction temperatures may be greater than or equal to 150° C., e.g., greater than or equal to 175° C. or greater than or equal to 185° C. In terms of ranges, the carbonylation reaction temperature may be from 150° C. to 250° C., e.g., from 175° C. to 230° C. or from 185° C. to 205° C. The carbonylation reaction is exothermic and temperature of the reactor may be regulated by a variety of methods. For purposes of the present disclosure, any suitable cooling may be used to regulate the temperature of the reactor. U.S. Pat. No. 5,374,774 describes a cooling unit in the recycle line for the reactor. A pump around loop may be used to generate additional heat for the production of steam while regulating the temperature of the carbonylation reactor, which is further described in U.S. Pat. No. 8,530,696. In some embodiments, the temperature of the reactor may be controlled by condensing a portion of the flash overhead that is returned to the reactor, which is further described in U.S. Pat. No. 8,957,248.

The production rate of acetic acid may be from 5 to 50 mol/L·h, e.g., from 10 to 40 mol/L·h, or from 15 to 35 mol/L·h. “Greater production rates” generally refers to operating above 20 mol/L·h.

Carbon monoxide is introduced at a rate sufficient to maintain the desired internal reactor pressure. In some embodiments, carbon monoxide is continuously introduced into the carbonylation reactor, desirably below the agitator, which may be used to stir the contents, and thoroughly disperse the carbon monoxide throughout the liquid reaction medium. Other methods of agitating the reaction medium may be employed, such as a vessel with an eductor or pump-around mixing, or bubble-column type vessel, with or without an agitator.

The material of the carbonylation reactor and its internals, including feed and effluent lines, are not particularly limited and may be a metal, a ceramic, a glass, or combinations thereof. For example, the material may include zirconium-based materials and alloys that tend to have high corrosion resistance, but may also include iron-based alloys (stainless steel), nickel-based alloys (HASTELLOY™ or INCONEL™), titanium-based materials and alloys, or aluminum-based materials or alloys.

Under continuous production conditions, various gas-phase components may be formed or evolved from the liquid reaction. The gas-phase component can include carbon monoxide, hydrogen, methane, carbon dioxide, acetic acid, methyl acetate, methyl iodide, hydrogen iodide, acetaldehyde, dimethyl ether, and water. In some embodiments, the gaseous stream contains low amounts of hydrogen iodide of less than or equal to 1 vol. %, e.g., less than or equal to 0.9 vol. %, less than or equal to 0.8 vol. %, less than or equal to 0.7 vol. %, or less than or equal to 0.5 vol. %. To prevent an undesirable buildup of various gas-phase components, a gaseous stream 12 would be withdrawn or vented the gaseous stream from the upper portion of the reactor.

Venting gaseous stream 12 from the reactor 10 further reduces the buildup of gaseous byproducts and maintains a set carbon monoxide partial pressure at a given total reactor pressure. To prevent loss of useful components, the gaseous stream 12 may be cooled by a heat exchanger with a cooling medium in one or more condensers to partially condense any condensable liquids present as vapors in the gaseous purge stream into a condensate portion and a gaseous portion. Condensate portion typically includes useful liquid products, such as acetic acid, methyl acetate, methyl iodide, acetaldehyde, dimethyl ether, water, or mixtures thereof, and is returned to the reactor. Although the gaseous portion may be flared, the gaseous portion typically includes sufficient amounts of carbon monoxide, hydrogen, methane, carbon dioxide and minor amounts of iodides such as methyl iodide or hydrogen iodide to make further recovery desirable.

The gaseous stream 12 may be further processed in an absorption system 90, such as a scrubber system or a pressure swing absorption tower. As typically done the gaseous stream 12 is condensed and the gaseous portion (noncondensable fraction) may be fed to the absorption system 90. In some embodiments, the gaseous portion owing to its relatively high carbon monoxide content is useful to stabilize the catalyst against precipitation. Absorption system 90 is capable of collecting and/or recovering useful components, in particular organic components as well as methyl iodide. A chilled solvent is fed via line 93 at the top of absorption unit 90 to recover such components in the residue 92, which may be returned to the reactor. The chilled solvent may comprise acetic acid, methanol, methyl acetate, water, or mixtures thereof, and is chilled to a temperature of less than or equal to 20° C., e.g., less than or equal to 18° C. or less than or equal to 10° C. Any remaining gaseous not collected in the residue 92 leave the absorption system 90 near the top via line 91. Although one absorber is shown for the absorption system 90, the absorption system may comprise multiple absorption towers as well as solvent stripping columns. Further, other vent streams obtained throughout the process may be collected and passed through the absorption system 90.

One absorption system involves multiple absorbing steps, e.g., with differing absorption solvents and/or differing pressures. Such systems are described in U.S. Pat. No. 8,318,977, which is incorporated herein by reference in its entirety.

Returning to the reactor 10, the catalyst in the reaction medium plays the role of promoting the methanol carbonylation reaction. In commercial production, the metal catalyst does not activate methanol directly, so a more reactive methyl substrate (reactant) must be generated in situ. An iodide promoter, such as hydrogen iodide, converts the methanol into methyl iodide. However, since most of the reaction medium is acetic acid, the methanol is esterified to methyl acetate, which is activated by hydrogen iodide into methyl iodide.

The components of the reaction medium are maintained within defined limits to ensure sufficient production of acetic acid and utilization of reactants, while limiting the production of byproducts. The following amounts are based on the total weight of the liquid phase of the reaction medium. In a continuous process, the amounts of components are maintained within the ranges provided and fluctuations within these ranges are anticipated. One of ordinary skill would readily understand how to control the process to maintain the amounts of components in the reaction medium.

The reaction medium includes a Group VIII metal catalyst, e.g., cobalt, rhodium, iridium, or combinations thereof, in an amount from 200 to 3000 ppm based on the metal in the reaction medium, e.g., from 800 to 3000 ppm, or from 900 to 1500 ppm. The metal catalyst may be a homogeneous catalyst, may be a resin-supported catalyst, or may be supported in a fixed bed.

Water in the reaction medium is a useful component for forming acetic acid according to the methanol carbonylation reaction mechanism, and further dissolves soluble components in the reaction medium. The mass composition of water in the reaction medium is maintained to be less than or equal to 14 wt %, e.g., from 0.1 wt % to 14 wt %, from 0.2 wt % to 10 wt % or from 0.25 wt % to 6 wt %, or from 1.8 to 4.1 wt %. To control the water mass composition, water may be continuously fed to the carbonylation reactor 10, including through the recycles lines, at a predetermined flow rate. In some embodiments, the reaction is conducted under low water conditions and the reaction medium contains water in an amount from 0.1 to 4.1 wt %, e.g., from 0.1 to 3.1 wt % or from 0.5 to 2.8 wt %. In another embodiment, the reaction is conducted with water in an amount of less than or equal to 2 wt % water, e.g., from 0.1 to 2 wt %, or from 0.1 to 1.9 wt %.

The promoter in the reaction medium may be an iodide to assist the activity of the catalyst. Non-limiting examples of the iodide as the promoter include methyl iodide, an ionic iodide, and combinations thereof. The mass composition of methyl iodide in the reaction medium is maintained to be from 1 to 25 wt %, e.g., from 5 to 20 wt %, or from 4 to 13.9 wt %. The ionic iodide can stabilize the metal catalyst and inhibit side reactions. Non-limiting examples of the ionic metal iodides include lithium iodide, sodium iodide, and potassium iodide. The mass composition of iodide metal iodides (ionic salts), e.g., lithium iodide, in the reaction medium is maintained to be from 1 to 25 wt %, e.g., from 2 to 20 wt %, or from 3 to 20 wt %. The iodide salt may be formed in situ, for example, by adding lithium acetate, lithium carbonate, lithium hydroxide or other lithium salts of anions compatible with the reaction medium. In some embodiments, the process may maintain a mass composition of lithium acetate in the reaction medium from 0.3 to 0.7 wt %, e.g., from 0.3 to 0.6 wt %.

It will be generally recognized that it is the amount of iodide ion in the catalyst system that is important and not the cation associated with the iodide, and that at a given molar mass of iodide, the nature of the cation is not as significant as the effect of the iodide. Any metal iodide salt, or any iodide salt of any organic cation, or other cations such as those based on amine or phosphine compounds (optionally, ternary or quaternary cations), can be maintained in the reaction medium provided that the salt is sufficiently soluble in the reaction medium to provide the desired level of the iodide. When the iodide is a metal salt, preferably it is an iodide salt of a member of the group consisting of the metals of Group IA and Group IIA of the periodic table as set forth in the “Handbook of Chemistry and Physics” published by CRC Press, Cleveland, Ohio, 2002-03 (83rd edition). In particular, alkali metal iodides are useful, with lithium iodide being particularly suitable.

As described above, the methyl acetate may be formed by the reaction between acetic acid and methanol. The mass composition of methyl acetate in the reaction medium is maintained to be from 0.5 to 30 wt %, e.g., from 0.5 to 20 wt %, from 0.6 to 9 wt %, or from 0.6 to 4.1 wt %.

Acetic acid is the main product of the reaction and the mass composition of acetic acid in the reaction medium, which also functions as solvent, is generally in amount of greater than or equal to 30 wt %, e.g., greater than or equal to 40 wt % or greater than or equal to 50 wt %. The acetic acid in the reaction medium includes acetic acid previously charged into the reactor upon start-up.

In addition to the acetic acid product, various byproducts and/or impurities may also be generated in the reaction medium. On the basis of previous extensive studies byproducts and/or impurities, such as, but not limited to hydrogen, methane, carbon dioxide, formic acid, hydrogen iodide, acetic anhydride, acetaldehyde, crotonaldehyde, 2-ethyl crotonaldehyde, dimethyl ether, propionic acid, and alkyl iodides such as ethyl iodide, hexyl iodide, decyl iodide and dodecyl iodide (collectively included with methyl iodide as C₁-C₁₂ alkyl iodide), have been found to be present in the reaction medium. Hydrogen iodide is formed via the reaction mechanism of the methanol carbonylation reaction when the catalyst alone or in combination with the promoter as described above is used. The reaction medium may have acetaldehyde in amounts ranging from 0 to 1800 ppm of the total reaction medium, e.g., from 10 to 1600 ppm, from 50 to 1000 ppm, from 50 to 800 ppm, or from 100 to 400 ppm. The reaction medium may have hydrogen iodide in amounts ranging from 50 to 5000 ppm of the total reaction medium, e.g., from 100 to 3000 ppm, or from 200 to 2000 ppm. In some embodiments, the reaction medium may also include acetic anhydride. The reaction medium may have acetic anhydride in amounts ranging from 0 to 5000 ppm of the total reaction medium, e.g., from 0.01 to 3000 ppm, or from 0.1 to 1000 ppm.

In handling acetic acid there may be corrosion issues not only from the acetic acid itself but from other components, and in particular hydrogen iodide. In one embodiment, to prevent or reduce the corrosion caused by hydrogen iodide, potassium hydroxide may be introduced. Potassium hydroxide should be carefully used since potassium acetate may also be produced by reacting with acetic acid. These potassium compounds may be returned to the reactor 10 though recycle lines. To maintain efficient acetic acid production, the potassium levels (from all sources of potassium, including but not limited to, potassium hydroxide and potassium acetate) in the reaction 10 may be not more than 500 ppm, e.g., not more than 350 ppm, or not more than 200 ppm.

Byproducts may be controlled by regulating the reaction medium and, in addition, the byproducts may be removed by separation process as described further herein. For example, as described in U.S. Pat. No. 8,017,802, formic acid may be controlled by controlling the water content in the reactor and/or temperature of reactor resulting in a formic acid content in the acetic acid product that is less than 160 ppm, e.g., less than 140 ppm, or less than 100 ppm. Separation of byproducts may be limited by the associated costs. When the byproducts are not removed, especially higher boiling point components, the components can concentrate in the acetic acid product. Thus, it is useful to limit the production of byproducts in the reactor to reduce the need for separation. For example, in some embodiments, the propionic acid amounts in the acetic acid product may further be maintained below 250 ppm by maintaining the ethyl iodide amounts in the reaction medium at less than or equal to 750 ppm, e.g., less than or equal to 400 ppm, less than or equal to 350 ppm, or less than or equal to 300 ppm, without removing propionic acid from the acetic acid product.

Evaporator (20)

In steady state operations, the reaction medium is continuously withdrawn from the reactor 10 as stream 11 at a rate sufficient to maintain a constant level therein. To obtain the acetic acid product, the withdrawn reaction medium in stream 11 is fed to the subsequent downstream evaporator 20, which may be a flash evaporator, flash vessel (e.g., flasher) or a flash distillation. In some embodiments, a converter reactor (not shown) or a pipe reactor (not shown) can be employed in the flow path between the reactor and evaporator. A pipe reactor is described in U.S. Pat. No. 5,672,744 and is used to react the dissolved carbon monoxide in the reaction medium. Chinese Patent No. CN1043525C describes a converter reactor to allow the reaction to proceed to a greater extent prior to subsequent flashing. The converter reactor produces a vent stream comprising gaseous components which are typically scrubbed with a compatible solvent to recover components such as methyl iodide and methyl acetate. As described herein, the gaseous stream from the reactor 10 and converter can be combined or scrubbed separately and are typically scrubbed with either acetic acid, methanol or mixtures of acetic acid and methanol, to prevent loss of low boiling components such as methyl iodide from the process.

Evaporator 20 performs a flash evaporation or distillation step, referred to herein as flashing or evaporating, which may be performed in multiple vessels, to return the catalyst recycle stream 22 to the reactor 10 and separate a vapor acetic product 21 comprising acetic acid for further processing. In some embodiments, the flashing may be performed by decompressing the reaction medium in stream 11 with or without heating. Stream 11 may be tangentially fed through one or more feed ports in an upper portion as shown in U.S. Pat. No. 6,599,348. To direct the liquid portion downwards, a splash plate may be used in each of the feed ports. In some embodiments, the flashing may be carried out, either adiabatically or thermostatically, to produce a vapor temperature from 100° C. to 260° C., and a residual liquid temperature from 80° C. to 200° C. The internal pressure (gauge) of the evaporator 20 may be from 0.5 atm to 5 atm, e.g., from 0.5 atm to 3.5 atm, 0.5 to 2.5 atm, or from 0.5 to 1.5 atm.

Evaporator 20 may be a vertical evaporator having a torispherical, ellipsoidal, or hemispherical head. To allow maintenance or access, evaporator 20 may have one or more manways. The nozzle for stream 11 may be in the upper portion of evaporator 20, e.g., above the liquid level within the evaporator 20. There may be one or more nozzles (not shown) that introduce the reaction medium tangentially to further disengage the vapor portion. In some embodiments, evaporator 20 may have an upper portion with a larger cylinder diameter than the lower portion. Evaporator 20 should have large volume to allow the reaction medium in stream 11 that is fed thereto to be maintained in the evaporator 20 to vaporize the desired carbonylation products into the vapor acetic product 21, and prior to recycling the catalyst recycle stream 22. In one embodiment, a residence time in the evaporator 20 of about one minute or more is desirable, and in some embodiments, a residence time of at least about two minutes or more may be used.

The mass ratio of the vapor acetic product 21 to the catalyst recycle stream 22, which are separated from each other, may be from 10:90 to 50:50, e.g., from 20:80, from 24:76, from 26:74, from 30:70 or from 40:60. The vapor acetic product 21 comprises acetic acid, as well as methyl iodide, methyl acetate, water, permanganate reducing compounds (PRC's), and other byproducts or impurities. Dissolved gases in the reaction medium that enter the evaporator are concentrated into the vapor acetic product 21. The dissolved gases comprise a portion of the carbon monoxide and may also contain gaseous byproducts such as methane, hydrogen, and carbon dioxide.

To maintain or enhance catalyst stability and reduce or prevent catalyst precipitation in evaporator 20, a CO-containing purge may be introduced to the lower section, e.g., below the feed nozzle, of the evaporator 20 or into the catalyst recycle stream 22. The CO-containing purge may comprise no less than 60 wt % carbon monoxide, e.g., no less than 80 wt %, or no less than 90 wt %. The amount of CO-containing purge may be sufficient to dissolve carbon monoxide into the liquid held up in the lower portion of the evaporator 20. In one embodiment, the CO-containing purge may be fed in an amount of greater than 5 Nm³/hr, e.g., greater than 50 Nm³/hr or greater than 100 Nm³/hr. While there is no upper limit for purposes of the present disclosure, a practical upper limit may be set at no more than 1000 Nm³/hr.

Even when CO-containing purges are used to stabilize the catalyst, there may be some insoluble forms that precipitate onto the interior surface. Owing to its relative expense, insoluble forms of rhodium that collect on the interior surface may be recovered for reuse.

In operating the process continuously, there may be some catalyst loss, thus necessitating the use of makeup catalyst. Although makeup catalyst may be added directly to reactor 10, in one embodiment, the makeup catalyst may be added to the evaporator 20 or to catalyst recycle stream 22. The makeup catalyst may be metered at a rate sufficient to maintain the continuous reaction.

In some embodiments, an optional mist eliminator may be employed near the vapor outlet to coalesce liquid droplets. An optional scrubbing section (not shown) may further be employed in the vapor outlet of the evaporator to prevent and/or reduce entrainment from metallic catalysts or other metallic components into the vapor acetic product 21. A wash liquid may be introduced into the optional scrubbing section. In another embodiment, an in-line separator may be used in the line for the vapor acetic product 21 to impart a swirling motion and to allow entrained liquid to coalesce. The liquid may be drained back to evaporator 20 to reduce entrainment in vapor acetic product 21.

Vapor acetic product 21 comprises acetic acid, methyl iodide, methyl acetate, water, acetaldehyde, and hydrogen iodide. The evaporator 20 may be operated under conditions sufficient to vaporize at least 80% of the methyl iodide and methyl acetate, based on the total reaction medium, into the vapor acetic product 21. In some embodiments, vapor acetic product 21 comprises acetic acid in an amount from 45 to 75 wt %, methyl iodide in an amount from 20 to 50 wt %, methyl acetate in an amount of less than or equal to 9 wt %, and water in an amount of less than or equal to 15 wt %, based on the total weight of the vapor product stream. In another embodiment, vapor acetic product 21 comprises acetic acid in an amount from 45 to 75 wt %, methyl iodide in an amount from 24 to less than or equal to 36 wt %, methyl acetate in an amount of less than or equal to 9 wt %, and water in an amount of less than or equal to 15 wt %, based on the total weight of the vapor product stream. More preferably, vapor acetic product 21 comprises acetic acid in an amount from 55 to 75 wt %, methyl iodide in an amount from 24 to 35 wt %, methyl acetate in an amount from 0.5 to 8 wt %, and water in an amount from 0.5 to 14 wt %. In yet a further preferred embodiment, vapor acetic product 21 comprises acetic acid in an amount from 60 to 70 wt %, methyl iodide in an amount from 25 to 35 wt %, methyl acetate in an amount from 0.5 to 6.5 wt %, and water in an amount from 1 to 8 wt %. The acetaldehyde amounts in the vapor acetic product 21 may be in an amount from 0.005 to 1 wt %, based on the total weight of the vapor product, e.g., from 0.01 to 0.8 wt %, or from 0.01 to 0.7 wt %. Vapor acetic product 21 may comprise hydrogen iodide in an amount less than or equal to 1 wt %, based on the total weight of the vapor product stream, e.g., less than or equal to 0.5 wt %, or less than or equal to 0.1 wt %. The propionic acid, acetic anhydride, or formic acid, if present, may be present in amounts in vapor acetic product 21 in a reduced amount of less than 1 wt %, e.g., less than 0.5 wt %.

In some embodiments, the entire vapor product stream is directed to the distillation column 30 (which may be referred to a splitter column or light ends column) as a vapor crude product 21. This provides the thermal energy to separate the components in the distillation column 30. In some embodiments, there is provided a condenser (not shown) that cools and partially condenses a portion of the vapor crude product to efficiently remove heat generated by the exothermic carbonylation reaction. In these embodiments, a condensed portion formed by cooling may be passed through a heat exchanger prior to being transferred to the reactor 10. In other embodiments, the condensed portion may be forwarded to the distillation column 30 to debottleneck and increase capacity. The non-condensable gaseous portion from cooling in the condenser may be directed to the absorption system 90.

To handle the catalyst recycle stream 22 (which is a liquid recycle stream) in a manner that maintains flow rates, prevents equipment damage, and provides sufficient control, a vortex breaker (not shown) may be used near the liquid outlet of the evaporator 20. Catalyst recycle stream 22 comprises acetic acid, the metal catalyst, corrosion metals, as well as other compounds that remain without volatilization in the flashing step. The metal catalyst may be maintained in its soluble form. In some embodiments, liquid recycle stream comprises acetic acid in an amount from 60 to 90 wt %, metal catalyst in an amount from 0.001 to 0.5 wt %, corrosion metals (e.g., nickel, iron and chromium) in a total amount from 10 to 2500 ppm, lithium iodide in an amount from 5 to 20 wt %, methyl iodide in an amount from 0.5 to 5 wt %, methyl acetate in an amount from 0.1 to 5 wt %, water in an amount from 0.1 to 8 wt %, acetaldehyde in an amount of less than or equal to 1 wt % (e.g., from 0.0001 to 1 wt % acetaldehyde), and hydrogen iodide in an amount of less than or equal to 0.5 wt % (e.g., from 0.0001 to 0.5 wt % hydrogen iodide).

Prior to returning the catalyst recycle stream 22 to the reactor, a slip stream may pass through a corrosion metal removal bed, such as an ion exchange bed, to remove any entrained corrosion metals, such as nickel, iron, chromium, molybdenum and/or zinc, as described in U.S. Pat. No. 5,731,252, which is incorporated herein by reference in its entirety. Also, the corrosion metal removal bed may be used to remove nitrogen compounds, such as amines, as described in U.S. Pat. No. 8,697,908, which is incorporated herein by reference in its entirety.

Further, the catalyst recycle stream 22 may be mixed with other recycles streams from the distillation column, or any other condensed liquid stream, and the mixture is recycled to the reactor 10 of the reaction step. A pump may be used to return the catalyst recycle stream 22 and/or mixture to the reactor 10.

In some embodiments, the evaporation rate of the evaporation step (with or without heating) may be from 20 to 80% by mass, e.g., from 20 to 70%, from 25 to 75%, from 25 to 60%, from 25% to 50%, or from 30% to 40%.

In addition to the vapor acetic product 21 and catalyst recycle stream 22, which are the main exit lines for evaporator, a vent stream may be withdrawn from evaporator as needed. The vent stream may be directed to the absorption system 90.

First Distillation Column (30)

Useful acetic acid is contained within the vapor acetic product 21 and it is advantageous to further obtain a high quality product by removing reaction components and byproducts. In one embodiment, to further obtain a high quality product, vapor acetic product 21 is fed to a first distillation step performed in a first distillation column 30. First distillation column 30 may be referred to as a light-ends column or splitter column. To allow for separation, the first distillation column 30 may comprise a plate column, a packed column or combination thereof. In the embodiments that use a plate column, the theoretical number of plates may range from 5 to 80 plates, e.g., from 10 to 60 plates or from 15 to 50 plates.

As described herein, the distillation step performed in distillation column 30 is for further purifying the vapor crude product 21, and yields at least two of the following streams: an overhead 31, a bottoms 32, or an acetic acid stream 33, which is removed as a liquid side draw. Although not limiting, the acetic acid stream 33 is generally withdrawn from above the stripping section and/or above the introduction point of the vapor crude product 21 and below the reflux streams. Any stream withdrawn from the stripping section, which is below the introduction point, may be referred as a bottoms 32 in the first distillation column 30. The number of streams yielded may depend on the composition of the vapor crude product 21 and reflux composition. For example when there is low amounts of water, an overhead 31 and either a bottoms 32 or acetic acid stream 33 may be taken that contains the acetic acid product. Also low amounts of components that have a higher boiling point than acetic acid reduces the amount withdrawn through the bottoms 32.

For purposes of the present disclosure, the first distillation column 30 will be described with all three streams, but this should not be construed as limiting the invention.

Vapor crude product 21 is continuously introduced and subject to separation into an overhead 31, an acetic acid stream 33 (side stream), and a bottoms 32. Overhead 31 is withdrawn from above the enriching section of the first distillation column 30, e.g., near the top, and/or an upper position of the first distillation column 30. The overhead 31 may contain water and is rich in at least one of methyl iodide and/or acetaldehyde. The proportion of the overhead 31 may be about 20% to 60%, e.g., about 35% to 50%, of the vapor crude product 21. Acetic acid stream 33 is a side draw which mainly contains acetic acid is taken from a position below the upper position of the overhead stream and above the inlet position of the vapor stream 21. A liquid re-distributor or configured tray may collect liquid for the acetic acid stream 33. Acetic acid stream 33 contains mostly acetic acid. In some embodiments, there may be multiple ports on the side of distillation column 30 for obtaining the acetic acid stream 33. The proportion of the acetic acid stream 33 may be about 30% to 80%, e.g., about 40% to 70%, of the vapor crude product 21. For high production of acetic acid, distillation column 30 operates by pushing a relevant amount of the vapor crude product 21 in the acetic acid stream 33 over the overhead 31. Thus, the acetic acid stream 33 may be larger, based on mass flows, than overhead 31. The bottoms 32 may be withdrawn from a stripping or lower portion of the first distillation column and/or a lower position of the first distillation column 30. The proportion of the bottoms 32 may be about 0% to 10%, e.g., from about 0 to 5% or from about 0% to 3%, of the vapor crude product 21.

Overhead 31 preferably contains lower boiling components, such as water, methyl acetate, methyl iodide, and carbonyl impurities. The amount of water in the first overhead stream is generally greater than or equal to 5 wt %. The carbonyl impurities present in the overhead 31 include acetaldehyde as well as various by-products derived from acetaldehyde, such as crotonaldehyde and 2-ethyl crotonaldehyde.

Next, the acetic acid stream 33 (side draw) is taken as a major stream from the separation in first column 30. To necessitate purification, acetic acid stream 33 preferably comprises a higher amount of acetic acid, based on mass, than the vapor crude product 21. The amount of acetic acid may be greater than 80 wt %, e.g., greater than 85 wt % or greater than 90 wt %. Water mass compositions in the side draw may vary depending on the reflux ratio, but are generally from 0.5 wt % to 5 wt %, e.g., from 0.6 to 3 wt %, or from 0.7 to 2.8 wt %. Methyl iodide, although preferably concentrating in the overhead, may also be present in lower amounts in the acetic acid stream 33 of not more than 6 wt %, e.g., not more than 3 wt % or not more than 1.7 wt %. Similarly, methyl acetate mass compositions are lower in the acetic acid stream 33 than the overhead 31 and may be not more than 3 wt %, e.g., not more than 1.5 wt % or not more than 1.2 wt %.

A portion of acetic acid stream 33 may be condensed and returned to distillation column 30 to further enhance the purity of acetic acid stream 33 and reduce the amounts of other components, in particular iodides and water.

In one embodiment to achieve purification for a high quality product, acetic acid stream 33 is fed to distillation column 34. Distillation column 34 concentrates acetic acid in the lower portion and removes mainly water in the overhead, which thus can be referred as a drying or dehydrating column. Distillation column 34 may operate under conditions sufficient to reduce the water amount in the lower stream 35, which is taken a bottom stream or a stream near the bottom in the lower portion of distillation column 35, and thus enriches acetic acid over acetic acid stream 33. Any overhead stream 36 may be condensed and refluxed as necessary and the remainder returned to the reactor 10. The lower stream 35 may be further passed through an ion exchange resin vessel 37 to remove iodides. As effectively employed silver-exchanged cation ion-exchange resin bed are sufficient for removing iodide in lower stream 35. In some embodiments, there may additional separation units, including but not limited to distillation, separators, evaporators, extractors, or other separation vessels, positioned upstream of distillation column 34, between distillation column 34 and ion exchange resin vessel 37, or downstream of ion exchange resin vessel 37 to remove further impurities. The product may be collected and stored from stream 38 as high quality acetic acid product.

In one embodiment to sequester entrained catalyst, such as rhodium, from entering distillation column 34 there may be a resin bed (not shown) downstream of distillation column 30. The entrained catalyst may be in amounts of up to 100 ppb. This resin bed may operate to treat acetic acid stream 33 during steady state operations and may be taken off-line for reclamation. The resin bed downstream of distillation column 30 benefits from a nickel-based or zirconium-based metallurgy due to the corrosive nature of the stream 33. The resin bed may be a polymeric substrate which includes a polymer with nitrogen-containing heterocyclic repeat units, which includes, but is not limited to, vinyl pyrrolidone or vinyl pyridine resins. Such suitable resins are further described in U.S. Pat. No. 7,902,398, the entire contents and disclosures of which is hereby incorporated by reference.

Returning to first distillation column 30, bottoms 32 is a relatively smaller stream and contains acetic acid, water, and other components (methyl iodide, methyl acetate, methanol, entrained catalyst, and/or propionic acid). The amounts of the other components are generally less than the water amounts in the bottoms 32. The water in the bottoms 32 may range from 0.5 wt % to 5 wt %, e.g., from 0.6 to 3 wt %. The acetic acid in the bottoms 32 may range from 60 wt % to 99 wt %, e.g., from 80 to 98 wt %. The bottoms 32 may be returned continuously or as needed to either the reactor 10 and/or the evaporator 20.

The internal temperature of the first distillation column 30 varies with the internal pressure. Operating pressure from 0.6 to 3 atm (gauge), e.g., from 0.7 to 1.5 atm, may be employed. In one embodiment, with an internal pressure of about 1 atm, the first distillation column may have a column top temperature (overhead temperature) of from 50 to 180° C., e.g., from 70 to 170° C., from 80 to 160° C., or from 90 to 150° C. In some embodiments, the thermal energy (e.g., heat) for the distillation step in the first distillation column 30 is primarily provided by the vapor crude product 21. In some embodiments, when supplemental/incremental heat is required, a reboiler may be provisioned.

As shown in FIG. 1, overhead 31 may be condensed in one or more condensers (not shown) and the condensate collected in a receiver (decanter) 40. In one embodiment, the cooling is achieved by indirect condensers, using for example process or cooling water, and the condensate and non-condensate vapors are collected in the receiver. The conditions within the receiver 40 are maintained to favor the formation of phases (liquid-liquid separation) to improve the removal of impurities (primarily acetaldehyde). Under favorable conditions the first overhead 31 may separate (biphasically separate) in the receiver 40 to form an upper phase (aqueous phase) 41 and a lower phase (organic phase) 42. Typically, the upper phase 41 is enriched in water, and the lower phase 42 is enriched in methyl iodide. The phase separation should maintain two separate phases, without forming a third phase or emulsion between the phases. One or more vent streams (not shown) may be withdrawn from the receiver 40 as needed to remove the collected non-condensed gases. As with the other streams containing non-condensable gases described herein, in particular vent streams, may be treated by the absorber 90.

The average residence time of the condensed components in receiver may be greater than or equal to 1 minute, e.g., greater than or equal to 3 minutes, greater than or equal to 5 minutes, greater than or equal to 10 minutes. In some embodiments, the average residence time of the condensed components in receiver is less than or equal to 60 minutes, e.g., less than or equal to 45 minutes, less than or equal to 30 minutes, or less than or equal to 25 minutes. In addition, overhead decanter may be arranged and constructed to maintain a low interface level to prevent an excess hold up of methyl iodide. The phase separation temperature within the receiver may be not less than −5° C. and not more than 70° C., and preferably is from 20° C. to 45° C. In cases where the temperature at the time of separation is greater than 75° C. or less than −10° C., liquid-liquid separation may not be achievable.

The composition of the first overhead 31 may include water, methyl iodide, PRCs or other known compounds. The compositions of first overhead 31, which is biphasically separable, for typical components (acetaldehyde, methyl iodide, methyl acetate, acetic acid, and water, etc.) may be described, although the overhead and other process streams inevitably contain other minor components (including noncondensable gases and/or impurities) as described below. As used herein, each stream, including impurities, has a total amount of 100% by weight on the basis of weight.

Upper phase 41 primarily includes water typically with methyl acetate, acetic acid, hydrogen iodide, acetaldehyde, dimethyl ether, methanol, and lesser amounts of methyl iodide. Methyl iodide concentrates in lower phase which further includes methyl acetate, acetaldehyde, dimethyl ether, hydrogen iodide, methanol, and lesser amounts of water and acetic acid. Other byproducts and impurities, such as but not limited to hydrogen iodide, formic acid, dimethyl ether, and crotonaldehyde, may be present in insignificant amounts. 1,1-dimethoxyethane mass compositions in the upper and lower phases may be less than or equal to 2 wt %, e.g., less than or equal to 1 wt % or less than or equal to 0.5 wt %. In some embodiments, the acetaldehyde amounts in upper phase is larger, based on wt %, than lower phase. For example, the upper phase may have the following composition shown in Table 1.

TABLE 1
Upper Phase from Light Ends Overhead

| | Range | Preferred | More Preferred |
|---|---|---|---|
| | (Wt %) | Range (Wt %) | Range (Wt %) |
| Water | 40-80 | 50-75 | 55-75 |
| Methyl Acetate | 1-50 | 1-40 | 1-15 |
| Acetic Acid | 1-40 | 1-30 | 5-25 |
| PRC's (AcH) | <5 | <3 | 0.001-1 |
| Methyl Iodide | <10 | <5 | 0.01-3 |
| Methanol | <5 | <1 | 0.01-3.5 |
| Hydrogen Iodide | <1 | <0.5 | 0.001-0.5 |

As used in the tables provided in this disclosure, the symbol “<” is designated to mean “less than or equal to” or “up to” the value provided, and indicates that the component may be optional.

A sufficient reflux stream may be obtained from the upper or lower phase. In some embodiments, a portion of upper phase 41b may be refluxed into first distillation column 30. The reflux ratio (amount of upper phase reflux 41b/amount of the distillate of the upper phase 41a) of upper phase to first distillation column 30 is from 0.5 to 20, e.g., from 1 to 15, or from 1.5 to 12. When the lower phase 42b is refluxed, it may be refluxed with the upper phase 41b.

Lower phase, which is primarily methyl iodide, or portion thereof is returned (recycled) to the reactor 10. In some embodiments, a portion of lower phase may be refluxed alone or with the upper phase to the first distillation column 30. The specific gravity of lower phase may be from 1.3 to 2, e.g., from 1.5 to 1.8, from 1.5 to 1.75 or from 1.55 to 1.7. As described in U.S. Pat. No. 6,677,480, the measured specific gravity in lower phase may correlate to the methyl acetate amounts in the reaction medium. As specific gravity decreases, the methyl acetate amounts based on mass in the reaction medium increases. In some embodiments, receiver is arranged and constructed to maintain a low interface level to prevent an excess hold up of methyl iodide. For example, the lower phase may have the following composition shown in Table 2.

TABLE 2
Lower Phase from Light Ends Overhead

| | Range | Preferred | More Preferred |
|---|---|---|---|
| | (Wt %) | Range (Wt %) | Range (Wt %) |
| Methyl Iodide | 60-98 | 60-95 | 75-93 |
| Methyl Acetate | 0.1-25 | 0.5-20 | 0.7-15 |
| Acetic Acid | 0.1-10 | 0.5-10 | 0.7-10 |
| Hydrogen Iodide | <1 | <0.5 | 0.001-0.5 |
| Water | <3 | 0.05-1 | 0.01-1 |
| PRC's (AcH) | <5 | <3 | 0.05-0.5 |
| Methanol | <5 | <1 | 0.01-3.5 |

The ratio, based on weight, of the flow rate of the upper phase withdrawn from the receiver relative to that of the lower phase withdrawn from the receiver may be, for example, about 0.1/1 to 10/1 (e.g., about 0.3/1 to 3/1) and preferably about 0.5/1 to 2/1 (e.g., about 0.7/1 to 1.5/1).

Off-gases may be vented from the first distillation column 30 and/or receiver 40 as needed and directed to the absorber 90.

In a continuous process there may be variations in flow, which if left unregulated may cause disruptions and processing difficulties. To account for these variations the process may deploy a hold tank to buffer the streams between the first distillation column 30 and receiver 40, or after the receiver for either upper phase 41 or lower phase 42. When used the hold tank is sized to account for up to 20% variations in flow entering and leaving the receiver 40.

Second Distillation Column (50)

The process embodies, in addition to the liquid-liquid separation in receiver 40, an extraction step for bringing the first mixture (first overhead 31 or a portion thereof [either the upper phase 41a and/or lower phase 42a]) in contact with an extractant 54 (e.g., water) to separate into an acetaldehyde extract (second mixture). This allows, the first overhead 31 or a portion thereof in the extraction step to be brought directly in contact with water to give an acetaldehyde extract. The extraction step may be achieved in the second distillation column 50. The distillation may be conducted either as batch distillation or continuous distillation. To allow for separation, the second distillation column 50 may comprise a plate column, a packed column or combination thereof. In the embodiments that use a plate column, the theoretical number of plates thereof, may range from 1 to 100 plates, e.g., from 2 to 80 plates or from 5 to 75 plates.

In one embodiment, the first overhead 31 or a portion thereof (either the upper phase 41a and/or lower phase 42a) is collected and directed via a feed line 43 to the second distillation column 50 at a first location that is between the top and bottom of the second distillation column 50. The composition of feed line 43, which may be derived from first overhead 31 and is referred to herein as the first mixture, contains a suitable amount of permanganate reducing compounds, including but not limited to acetaldehyde, to be removed, and may have mass compositions of such components in an amount from 0.05 to 50 wt %, from 0.05 to 10 wt %, from 0.1 to 5 wt % or from 0.1 to 1 wt %. Thus, a targeted amount of permanganate reducing compounds may be separated from feed line 43. As used herein, the terms “mass composition of permanganate reducing compounds” or “permanganate reducing compounds mass composition” may be the total mass composition of all the permanganate reducing compounds or may be the mass composition of each permanganate reducing compounds. The representative permanganate reducing compound includes acetaldehyde.

The first mixture in feed line 43 may also contain other components, including but not limited to C₁-C₁₂ alkyl iodides (including methyl iodide), acetic acid, methyl acetate, water, and/or methanol, and others. When a mixed composition is derived from portions of the lower and upper streams, the first mixture may have a mass composition of C₁-C₁₂ alkyl iodides that is from 2.5 wt % to 90 wt %, e.g., from 10 wt % to 85 wt %, or from 20 to 70 wt %, and water that is from 0.5 wt % to 90 wt %, e.g. from 1 wt %, to 90 wt %, or from 1.5 wt %, to 85 wt %, in addition to the permanganate reducing compounds. A representative C₁-C₁₂ alkyl iodide is methyl iodide. In one embodiment the mass composition of C₁-C₁₂ alkyl iodides is greater than the mass composition of water. Importantly, in the feed line 43 there is a composition where the mass composition of permanganate reducing compounds, C₁-C₁₂ alkyl iodides, and water can be selected from a wide range disclosed herein. The composition in feed line 43 may be a homogeneous liquid or a mixture of lower and upper streams. The composition in feed line 43 may also comprise methyl acetate in an amount up to 30 wt %, e.g. from 0.1 to 28 wt %, or from 1 to 20 wt %, acetic acid in an amount up to 25 wt %, e.g., from 0.01 to 12 wt %, or from 0.5 to 7.5 wt %, and dimethyl ether in an amount up to 1 wt %, e.g., from 0.001 to 1 wt %, or from 0.004 to 0.8 wt %.

In a case where the feed line 43 contains the lower phase 42a from the receiver 40, the feed line 43 may have the composition shown in Table 2 above. Although not necessarily preferred, in a case where feed line 43 contains the upper phase 41b from the receiver 40, the feed line 43 may have the composition shown in Table 1 above.

In addition to these components, the mixed composition also may comprise methanol. The methanol may be unreacted methanol, or methanol obtained through secondary reactions in during the separation and/or distillation process. In one embodiment, the first mixture has a methanol mass composition that is less than the difference based on mass between the one or more C₁-C₁₂ alkyl iodides and water. When the mass composition of water exceeds the mass composition of the one or more C₁-C₁₂ alkyl iodides, the methanol mass composition may be less than the difference between water and the one or more C₁-C₁₂ alkyl iodides and in some embodiments, may be zero. In one embodiment, the mass composition of one or more C₁-C₁₂ alkyl iodides may be increased by deriving the first mixture from the lower phase 42, which predominately contains methyl iodide. Thus, water mass composition may be controlled by using less of the upper phase 41, including none of the upper phase 41, for the first mixture in feed line 43. Thus, particular weight ratios of lower phase 42 to upper phase 41 for the first mixture may range from 100:0 to 10:90, e.g., from 100:0 to 40:60, from 100:0 to 60:40 or from 100:0 to 80:20. This controls the amount of methanol that is introduced to the second distillation column 50. In one embodiment, the methanol mass composition in the first mixture may be less than or equal to 2 wt %, e.g., less than or equal to 1.8 wt %, less than or equal to 1.5 wt %, less than or equal to 1.1 wt %, less than or equal to 1.0 wt %, or less than or equal to 0.5 wt %. A lower limit of methanol in the first mixture may be more than 0.01 wt %, e.g., more than 0.05 wt % or more than 0.1 wt %.

Second distillation column 50 operates to separate a second mixture, which may be withdrawn as an overhead stream 51 or sidedraw 53, and a lower stream 52. In some embodiments, the second mixture comprises overhead stream 51 (or a portion thereof) and/or sidedraw 53 (or a portion thereof). The extraction in second distillation column 50 yields a second mixture that contains significantly more acetaldehyde than the feed line 43. Thereby the ratio, on a weight basis, of acetaldehyde to methyl iodide in the second mixture is greater than the acetaldehyde to methyl iodide ratio in the first mixture. Second mixture contains acetaldehyde, which is the principal PRC, which has been distilled or processed in second distillation column 50 and may be subsequently processed to remove acetaldehyde from the process or reduce the amount of acetaldehyde, as described further in detail. Second mixture may be withdrawn higher than, nearer to the top of the second distillation column 50, than that of the first location. To provide rectification, in one embodiment, there may be at least one or more actual plates between the first location and the withdrawn location of the second mixture.

In one embodiment, acetaldehyde may be separated from methyl iodide in an efficient process that reduces the loss of methyl iodide, and may function or operate even when first mixture is made to comprise methyl acetate, methanol, acetic acid, or combinations thereof. In particular, acetaldehyde is separated from the first mixture to reduce the amount of acetaldehyde in the lower stream 52. Lower stream 52, owing to the relative amounts of methyl iodide and methyl acetate, may be returned as a liquid recycle (directly or indirectly) to the reactor 10. Indirect recycle refers to a process whereby these streams passes through another vessel which may further process the lower stream 52 prior to returning to the reactor 10. Direct recycling may include combining the recycle stream with one or more other recycle streams.

Even though acetaldehyde mass composition in lower stream 52 is reduced or suppressed, it is also useful to control reaction byproducts of acetaldehyde, which may be converted to acetaldehyde upon recycling. In particular, one byproduct of acetaldehyde that tends to form in the extractive step of the second distillation column 50 may be an acetal or hemiacetal, which includes but is not limited to 1,1-dimethoxyethane. Through various secondary or side reactions with methanol, acetaldehyde may be converted to 1,1-dimethoxyethane. Owing to the relatively higher boiling point of acetals as compared to the aldehydes, the acetals fall into the lower stream 52, which is subsequently recycled. 1,1-dimethoxyethane has a boiling point of 64° C. The formation of acetals and/or hemiacetals reduces the efficiency of second distillation column, because instead of effectively removing acetaldehyde, the acetal builds up in the lower portion and is returned to the reactor. Through reversible reactions, acetaldehyde may be yielded from the recycled acetal and this decreases the effectiveness of second distillation column 50. There are a number of factors that can lead to the increasing acetal amounts, which includes when excessive methanol is introduced to the second distillation column 50, either in the first mixture or separately. Temperature and reflux ratio of the second distillation column 50 may also influence the acetal amounts.

Thus, in one embodiment, lower stream 52 is withdrawn from the second distillation column 50 when at least one of the following conditions (i) to (iii) are satisfied. Condition (i) is when the total acetal amounts in the lower stream is not more than 0.02 wt %. Condition (ii) is when the total acetal mass composition in the lower stream is less than the combined mass composition of methanol and acetaldehyde in the lower stream. Condition (iii) is when the total acetal mass composition in the lower stream is less than the methanol mass composition in the first mixture. It is contemplated that first mixture may contain no methanol and in such embodiments, condition (iii) may be met when the lower stream contains no acetals. Although a process that satisfies all three conditions is within the scope and preview of the present disclosure, it is not necessary to satisfy all three conditions to maintain lower mass compositions of acetal. In some embodiments, two of the conditions may be met such as condition (i) and condition (ii) or condition (i) and condition (iii). In other embodiments, one of the conditions may be met.

Second distillation column 50 may be fed with an extractant having a suitable composition for extracting acetaldehyde from the mixture, which is shown as the extractant 54 in FIG. 1. The extractant may be suitable composition for extracting acetaldehyde from the first mixture. For ease of processing, the extractant may also be separable from methyl iodide using a low energy technique including liquid-liquid separation and/or membrane separation. The extractant may comprise extracting water, a mixed solvent or a water-soluble organic solvent (glycol, glycerin, acetones, ethers, and/or esters). Introducing water is advantageous to maintain the extracting mixture in a liquid-liquid separation state and thus, the extractant is made to comprise not less than 80 wt % water, e.g., not less than 90 wt % water, or not less than 95 wt % water. In one embodiment, to prevent excess formation, the extractant practically does not comprise methanol, or other mono-alcohols. Thus, additional methanol is not fed into the second distillation column 50. Despite water or a water-containing extractant being preferred extracting mixtures, in some embodiments, second distillation column 50 may distill the mixed composition of feed line 43 without additional water.

In one embodiment, the water supply may be a fresh source of water external to the process. In another embodiment, the water supply for the extractant may be an internal stream containing water that is redirected to the top of the second distillation column 50. Any suitable internal stream may be used, including the upper phase 41, the overhead 36 of the distillation column 34, or the water extractant from a solvent recovery column. In one embodiment, internal streams that are deficient in methanol are preferred to be used as the extractant, including internal streams having a methanol mass composition of less than or equal to 0.1 wt %. When the internal streams contain higher amounts of methanol these internal streams may be treated to reduce the methanol amounts in a suitable manner. Any acetaldehyde or other PRC contained in these internal streams may be recovered in the second mixture.

As shown in FIG. 1, second distillation column 50 separates the components of feed line 43 through extractive distillation. In one embodiment, the feed line may be extracted with an extractant 54 that is introduced in an upper zone of the second distillation column 50 is above the first location. In one embodiment, extractant 54 may be introduced in top of the second distillation column 50, separate from the first location. Thus, in embodiments where the extractant 54 is made to comprise a portion of the upper phase 41, the upper phase 41 is fed above and separately from the feed line 43. The amount of PRC is higher in the upper zone and introducing the extractant 54 to this zone yields an extractant mixture, which is withdrawn as a second mixture (either an overhead 51 or sidedraw 53) that is enriched in acetaldehyde, as well as other PRCs. A relatively lower amount of extractant may be used when the PRCs, including acetaldehyde, are extracted into the second mixture (overhead or sidedraw) as opposed to the lower stream. For example, the flow rate ratio (on a weight basis) of the extractant 54 relative to first mixture in line 43 may range from 0.0001/100 to 100/100, e.g. 0.001/100 to 50/100, 0.0001/100 to 20/100, 0.001/100 to 10/100, 0.01/100 to 8/100, or 0.1/100 to 5/100.

In using an extractive distillation step in the second distillation, PRC's and the C₁-C₁₂ alkyl iodides may be processed in an efficient manner that reduces the energy requirements of the process.

Extractive distillation allows the number of stages for the second distillation column 50 to be reduced. In one embodiment, second distillation column has less than or equal to 100 stages, e.g., less than or equal to 80 stages or less than or equal to 45 stages. To illustrate the feed locations and the location of the withdrawn second mixture, the following example is provided using 50 stages, where lower stream (bottom) 52 is the 0^{th} plate, and the overhead 51 is the 50^{th} plate. These illustrative examples can be applied to a distillation column having a different amount of stages or plates. The first location, where the feed line 43 is introduced may be selected in the range from the 1^{st} plate to the 35^{th} plate, e.g., from the 3^{rd} plate to the 25^{th} plate or from the 5^{th} plate to the 20^{th} plate. The second location for withdrawing the sidedraw, may be selected in the range from the 20^{th} to 49^{th} plate, e.g., from the 25^{th} to 48^{th} plate or from the 35^{th} to 48^{th} plate, provided that the second location is withdrawn from a plate, such as a collector tray, above the first location. In one embodiment, there may be at least 1 plate separating the second location and first location, e.g., at least 5 plates of separation. Although the extractant 54 may be added at the top, e.g., 50^{th} plate, in some embodiments, the extractant may be added a location selected in the range from the 45^{th} to 50^{th}, e.g., from the 45^{th} to 49^{th} plate.

The extraction efficiency may increase by countercurrently adding the extractant 54 to the upper zone. Further, the extractant 54 may be added by spraying or sprinkling or any other suitable method, including adding in a droplet form. In one embodiment, the extractant 54 may have a temperature from 0° C. to 60° C., e.g., 10° C. to 50° C., and 20° C. to 40° C. In other embodiment, the process may involve pre-heating or warming of the extractant to a temperature from 30° C. to 150° C., e.g., 50° C. to 110° C. or from 60° C. to 110° C.

As shown in FIG. 1, second distillation column 50 is provided with a collector tray (plate) 55, which may be referred to as a hat tray or chimney tray, to allow the good vapor distribution to the upper zone from the feed line 43 and the holding of the whole amount of the extraction mixture to be taken off as sidedraw 53. Any suitable design for the collector tray 55 may be used with the embodiments described herein. The collector tray 55 is practically located where the sidedraw 53 is taken and thus the extractant 54 is be added above the collector tray 55. This allows the falling liquid from the upper portion of the second distillation column 50 to be received on collector tray 55. Although not shown in FIG. 1, in a case where the collector tray 55 is disposed at a position lower than the feed location of the mixed composition, the collector tray 55 is positioned above the lower stream 52.

In one embodiment, the second distillation column 50 is provided with at least one collector tray 55. For a distillation column having a plurality of collector trays, the extractant 54 may be added above uppermost collector tray.

In one embodiment, the second distillation column 50 is provided with a collector tray, the extractant or extracting water is added to the upper zone above the collector tray 55. The upper zone is formed in a space between the first location and the top of the distillation column 50. Acetaldehyde has a relatively low boiling point as a PRC and methyl iodide has a relatively low boiling point as a C₁-C₁₂ alkyl iodide, which forms, in the upper zone, a second mixture. In one embodiment, the collector tray 55 may be disposed at an upper position of the second distillation column 50. The PRCs are extracted efficiently by withdrawing the sidedraw 53 from the upper zone, and thus the position of the collector tray 55 is practically upper than the first location for the feed line 43 (mixed composition). The collector tray 55 is not limited to a particular position, and may be disposed at the same height level as the height level of the first location or may be disposed in a lower zone below the first location.

The height level of the collector tray 55 in the second distillation column 50 is upper than the first location of the feed line 43. In cases when a collector tray 55 is used, the collector tray may replace a plate in the distillation column. According to the number of plates of the second distillation column, the height level of the collector tray 55 is in between the uppermost plate of the column (the 1st plate from the top of the column) and a plate at least one plate upper than the feed line 43 or is positioned at or near the top of the second distillation column. In a case where the total number of plates of the distillation column is 50, the height level of the collector tray 55 may be selected from the range corresponding to the 1^{st} to the 35^{th} plate from the top of the distillation column, e.g., from 2^{nd} to 30^{th} plate, or from 2^{nd} to 15^{th} plate. These illustrative examples can be applied to a distillation column having a different amount of stages or plates.

The operating conditions for the second distillation column 50 maintain the extractive environment for recovering the second mixture. As understood the internal temperature of the second distillation column 50 is related to the column top pressure (absolute). This pressure is generally controlled or maintained within pressure from 100 kPa to 500 kPa, e.g., from 100 kPa to 250 kPa, or from 100 kPa 100 to 200 kPa. At atmospheric pressure (approximately 101 kPa), the second distillation column operates with a top temperature from 15° C. to 120° C., e.g., from 15° C. to 100° C. or from 15° C. to 80° C., and bottom temperature from 35° C. to 165° C., e.g., 40° C. to 150° C., or 40° C. to 115° C. In one embodiment, the bottom temperature is maintained above a temperature that maintains acetaldehyde in a gaseous state below the collector tray 55 to drive the acetaldehyde towards the upper zone for extracting into the second mixture.

Depending on the mixed composition in feed line 43, acetaldehyde may be effectively extracted into the second mixture, notwithstanding that presence of methyl acetate and/or acetic acid, which tend to have an affinity with both PRCs (including acetaldehyde) and C₁-C₁₂ alkyl iodides (including methyl iodide). In one embodiment, the amount of acetaldehyde in the second mixture (including the overhead 51 and/or sidedraw 53) is from 5 to 1000 times more than the amount in the first mixture on a mass basis, e.g., from 10 to 500 times or from 20 to 300 times.

In one embodiment, the second mixture is taking from the sidedraw 53 and the overhead 51 is condensed and refluxed at the top of the second distillation column 50. In some embodiments, there may be a distillate that is removed from the top of the second distillation column 50, but generally the condensed portion of the overhead 51 is refluxed. The overhead 51 exits second distillation column 50 with a temperature from 15° C. to 120° C. and the condenser (or plurality of condensers as needed) may condense the overhead 51 to a temperature lower than the boiling point of methyl iodide. The condensed liquid 57 is accumulated in an overhead receiver 56 and refluxed via line 58. To maintain extractive conditions, line 58 may enter second distillation column 50 between the location of the extractant 54 and withdrawing location of sidedraw 53 (e.g., above collector tray 55). This reflux can be used to prevent excess amounts of extractant and namely water, from being present in the overhead 51.

In a lower portion of second distillation column 50 a miscible solvent may be directly or indirectly fed. This solvent is miscible with a process stream containing methyl iodide. The miscible solvent may be at least one component selected from the group consisting of water, acetic acid, methyl iodide, and methanol. When added, the miscible solvent may be not more than 30 wt % relative to the amount of the sidedraw 53 withdrawn from collector plate 55, e.g., not more than 15 wt %, or not more than 10 wt %.

The composition of overhead 51 in general has a total mass composition of PRC and C₁-C₁₂ alkyl iodides that exceeds than the total mass composition of the remaining organic components. In one embodiment, the composition of the overhead 51 may have a PRC mass composition from 1 to 75 wt %, e.g., from 5 to 65 wt % or from 10 to 50 wt %, and C₁-C₁₂ alkyl iodides mass composition from 1 to 85 wt %, e.g., from 10 to 80 wt %, or from 20 to 60 wt %. The remaining organic components may comprise methyl acetate, acetic acid, methanol, and dimethyl ether, and are in various amounts which can include amounts of less than or equal to 10 wt %, e.g., less than or equal to 5 wt %. The water mass composition in the overhead 51 may be less than the water in the first mixture in feed line 43. The overhead 51 may have a water mass composition that is not more than 20 wt %, e.g., not more than 15 wt %, not more than 10 wt % or not more than 5 wt %. The overhead 51 may have a methyl acetate mass composition of up to 10 wt %, e.g., up to 5 wt %, or up to 1 wt %. The overhead 51 may have an acetic acid mass composition of up to 10 wt %, e.g., up to 5 wt %, or up to 1 wt %. Generally it is desired to have the lowest possible acetic acid mass composition by reducing the acetic acid in the feed line 43. Likewise the mass compositions of dimethyl ether and methanol in the overhead may also be maintained at low levels. The mass composition of dimethyl ether in the overhead may be not more than 1.8 wt %, e.g., not more than 1.5 wt %, or not more than 1 wt %, and the mass composition of methanol in the overhead may be not more than 0.55 wt %, e.g., not more than 0.35 wt % or not more than 0.25 wt %.

In some embodiments, the second mixture may be the overhead 51 or may comprise a portion of the overhead 51. In some embodiments, the overhead 51 is condensed and a distillate portion is used as the second mixture for removing PRCs. In the embodiment shown in FIG. 1, the composition of overhead 51 may be phase separable in overhead receiver 56 into an aqueous phase 59 and an organic phase 58. The organic phase 58 may be enriched in methyl iodide and deficient in water, while the aqueous phase 59 may contain useful amounts of PRCs and water.

The condenser used for condensing overhead 51 also produces a noncondensed portion which may be removed from the condenser or from the overhead receiver 56. Regardless of removal, the noncondensed portion may be further treated in absorption unit 90 to recover any organic components. This noncondensed portion may contain gaseous components such as but not limited to one or more of carbon oxides, nitrogen, hydrogen iodide, other inerts, and/or oxygen. The organic portion in the noncondensed portion is similar to the condensed portion described above, except the amount of dimethyl ether may be significant higher, e.g. not more than 90 wt %.

Sidedraw 53 (second mixture) is withdrawn as a liquid stream from distillation column 50. As a result of the extractive nature of the distillation column 50, sidedraw 53 may have a ratio, based on weight, of PRC to alkyl iodides, that is greater than the ratio of PRC to alkyl iodides in the first mixture. This further concentrates PRC for removal. The process according to the present invention concentrates the PRC without building up large quantities of acetals. In one embodiment, the composition of the sidedraw 53 (second mixture) may have PRC in amounts ranging from 0.1 to 90 wt %, e.g., from 0.2 to 65 wt % or from 0.5 to 50 wt %, C₁-C₁₂ alkyl iodides in amounts ranging from 0.5 to 95 wt %, e.g., from 1 to 95 wt %, from 5 to 90 wt %, or from 10 to 60 wt %, methyl acetate in amounts ranging from 0.1 to 25 wt %, e.g., from 0.5 to 20 wt %, or from 0.5 to 10 wt %, acetic acid in amounts ranging from 0 to 10 wt %, e.g., from 0.01 to 5 wt %, or from 0.05 to 1 wt %, water in amounts ranging from 0.1 to 20 wt %, e.g., from 0.5 to 15 wt %, or from 0.5 to 8 wt %, methanol in amounts ranging from 0 to 2.5 wt %, e.g., from 0.01 to 2.1 wt %, or from 0.05 to 2 wt %, acetal in amounts ranging from 0 to 2.5 wt %, e.g., from 0.01 to 1.7 wt %, or from 0.05 to 1.5 wt %, and dimethyl ether in amounts ranging from 0 to 1.2 wt %, e.g., from 0.01 to 0.8 wt %, or from 0.05 to 0.5 wt %.

As shown in FIG. 1, the second mixture is collected in vessel 60. Vessel 60 may be a buffer tank or may be a liquid-liquid separation vessel capable of receiving the second mixture and separating the second mixture into phases. In some embodiments, vessel 60 separates the liquid-liquid separable second mixture into an aqueous phase 61 and an organic phase 62. PRCs, including acetaldehyde, distributes more favorably into the aqueous phase 61 than organic phase 62. In addition, the extractant is more favorably separated into aqueous phase 61, and the extractant may be recovered through subsequent processing of the aqueous phase, although it is not necessary to recover the extractant. The organic phase may be returned to second distillation column 50 or combined with the lower stream 52 and is returned to the reactor 10. In addition, it is desirable to have reduced amounts of methyl iodide in the aqueous phase 61 so that the acetaldehyde may be discharged without further processing.

In one embodiment, the vessel 60 separates the second mixture 53 into an aqueous phase 61 and organic phase 62. The mass flow ratio of the aqueous phase 61 and the organic phase 62 may be from 1:1000 to 1:1 (aqueous phase to organic phase), e.g., from 1:900 to 1:10 or from 1:650 to 1:100. On balance, the aqueous phase may be the smaller stream, based on the mass flow, than the organic phase. The organic phase 62 is deficient in the extractant and may be returned to the second distillation column 50.

The aqueous phase 61 has higher PRCs (acetaldehyde) amounts than the organic phase 62, based on mass, and the aqueous phase 61 may have a higher amounts of PRCs than C₁-C₁₂ alkyl iodides (methyl iodide). Using acetaldehyde and methyl iodide as representatives, the aqueous phase 61 may have a ratio of former to latter, on a weight, from 2:1 to 60:1, e.g., from 3:1 to 45:1, from 3:1 to 30:1, or from 4:1 to 20:1. The composition of aqueous phase 61 comprises a PRC (acetaldehyde) mass composition from 1 to 50 wt %, e.g., from 5 to 45 wt % or from 10 to 35 wt %, water mass composition from 40 to 95 wt %, e.g., from 50 to 90 wt %, or from 60 to 75 wt %, C₁-C₁₂ alkyl iodides (methyl iodide) mass composition from 0.01 to 15 wt %, e.g., from 0.1 to 10 wt %, or from 0.5 to 6 wt %, methyl acetate mass composition from 0.1 to 25 wt %, e.g., from 0.5 to 20 wt %, or from 0.5 to 10 wt %, acetic acid mass composition from 0 to 5 wt %, e.g., from 0.01 to 2.5 wt %, or from 0.05 to 1 wt %, methanol mass composition from 0 to 2.5 wt %, e.g., from 0.01 to 2.1 wt %, or from 0.05 to 2 wt %, acetal mass composition from 0 to 2.5 wt %, e.g., from 0.01 to 1.7 wt %, or from 0.05 to 1.5 wt %, and dimethyl ether mass composition from 0 to 1.2 wt %, e.g., from 0.01 to 0.8 wt %, or from 0.05 to 0.5 wt %.

The organic phase 62 may be returned to second distillation column 50 below the collector tray 55. In one embodiment, the composition of organic phase 62 comprises a C₁-C₁₂ alkyl iodides (methyl iodide) mass composition from 0.1 to 90 wt %, e.g., from 5 to 85 wt %, or from 10 to 80 wt %, methyl acetate mass composition from 0.1 to 30 wt %, e.g., from 0.5 to 20 wt %, or from 0.5 to 10 wt %, PRC (acetaldehyde) mass composition from 0.01 to 15 wt %, e.g., from 0.5 to 10 wt % or from 0.5 to 5 wt %, acetic acid mass composition from 0 to 5 wt %, e.g., from 0.01 to 2.5 wt %, or from 0.05 to 1 wt %, water mass composition from 0.01 to 5 wt %, e.g., from 0.05 to 4 wt %, or from 0.5 to 3.5 wt %, methanol mass composition from 0 to 2.5 wt %, e.g., from 0.01 to 2.1 wt %, or from 0.05 to 2 wt %, acetal mass composition from 0 to 2.5 wt %, e.g., from 0.01 to 1.7 wt %, or from 0.05 to 1.5 wt %, and dimethyl ether mass composition from 0 to 1.2 wt %, e.g., from 0.01 to 0.8 wt %, or from 0.05 to 0.5 wt %.

To operate with effectively, the lower stream 52 contains a significant portion of the methyl iodide from first mixture, in particular when first mixture is made to comprise a portion of the lower phase 42a. The lower stream 52 of distillation column 50 contains useful methyl iodide that is returned to the reactor 10. To achieve production, the distillation column removes 60 to 99.9% of the methyl iodide in the first mixture into the lower stream 52, e.g., from 75 to 99.5% or from 80 to 99.1%. Successful removal of methyl iodide provides a lower stream 52 having a mass composition of C₁-C₁₂ alkyl iodides (methyl iodide) from 10 to 90 wt %, e.g., from 15 to 85 wt %, or from 20 to 80 wt %.

The process may withdraw a lower stream 52 that satisfies operating conditions (i) to (iii) described above to maintain acceptable acetal mass compositions. In one embodiment, the composition of the lower stream 52 may operate with condition (i) that is the total acetal mass composition in the lower stream 52 is not more than 0.02 wt %, e.g., not more than 0.018 wt %, not more than 0.015 wt % or not more than 0.01 wt %. In one embodiment, the C₁-C₁₂ alkyl iodides (methyl iodide) mass composition from 10 to 90 wt %, e.g., from 15 to 85 wt %, or from 20 to 80 wt %. Under conditions (ii), the combined mass composition of methanol and acetaldehyde may be not more than 2 wt %, e.g., not more than 1.9 wt %. The lower stream 52 may also comprise a methanol mass composition that is not more than 1.9 wt %, e.g., not more than 1.5 wt %, not more than 1 wt %, not more than 0.8 wt %, not more than 0.7 wt %, not more than 0.5 wt %, not more than 0.4 wt %, or not more than 0.3 wt %. The lower stream 52 may also comprise a PRC (acetaldehyde) mass composition is not more than 1.9 wt %, e.g., not more than 1 wt %, not more than 0.5 wt %, not more than 0.1 wt %, not more than 0.05 wt %, not more than 0.04 wt %, not more than 0.02 wt %, or not more than 0.005 wt %. In one embodiment, the water of the first mixture is transferred to the second mixture (overhead or side draw) more favorably than the lower stream 52. In general, the water mass composition in the lower stream 52 is less than 1.5 wt %, e.g., less than 1.1 wt %, less than 1.0 wt %, less than 0.7 wt %, or less than 0.5 wt %. Other components in the lower stream 52 may contain methyl acetate and dimethyl ether. Methyl acetate mass composition in the lower stream 52 may be from 5 to 60 wt %, e.g., 10 to 50 wt %, or 10 to 35 wt %, and dimethyl ether mass composition may be from 0 to 1.2 wt %, e.g., from 0.001 to 0.8 wt %, or from 0.005 to 0.5 wt %.

The lower stream 52 may be withdrawn at a temperature from 30° C. to 160° C., e.g., 35° C. to 120° C., or 40° C. to 100° C.

The lower stream 52 may be combined with at least a portion of organic phase 62.

Supplementary Acetaldehyde Removal

Although acetaldehyde, including other PRCs, are removed in the second mixture from the second distillation column 50, it may be desirable to remove or reduce acetaldehyde through supplementary processing and recover either useful organic components and/or extractant. There are several available methods for achieving such supplementary removal of acetaldehyde. For the purposes of present invention, these supplementary removal processes if used at all, can vary depending on the requirements on the processing facility.

In one embodiment, acetaldehyde may be removed or reduced by purging the second mixture from the process. This may be done with the second mixture that contains very low amounts of methyl iodide, in particular amounts that are less than 1 wt %, e.g., less than 0.5 wt %. When the second mixture contains higher amounts of methyl iodide it may be desirable to avoid purging of the second mixture by employing a supplemental acetaldehyde removal process.

In another embodiment, there may be a second extraction step of the second mixture in an extractor having no stages or distillation column having stages. For this supplemental acetaldehyde removal process, the second extraction uses a secondary extractant (additional water) and may yield an extractant containing the acetaldehyde and a raffinate containing the methyl iodide. This allows the raffinate to be recovered and the extractant to be further disposed of or purged. Under this arrangement, the second extraction may be positioned as a consecutive stage with the second distillation column 50. There may be a condenser/chiller between the extraction stages, i.e. the second distillation column 50 and extractor. The temperature of the second mixture using the condenser/chiller may be from 10° C. to 80° C., e.g., from 12° C. to 65° C. or from 13° C. to 45° C.

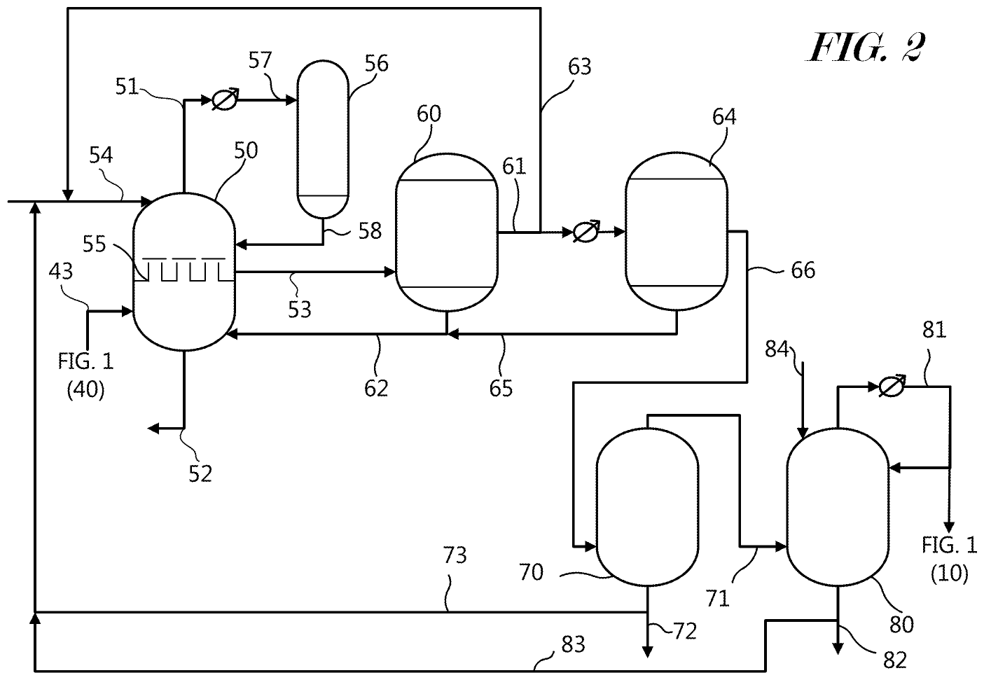

FIG. 2 is one embodied process of a supplemental acetaldehyde removal process. As shown in FIG. 2, the second mixture is withdrawn as sidedraw 53 from second distillation column 50 and introduced to vessel 60. The overhead 51 is condensed and the condensed portion 57 is reflux via line 58. Water is used an extractant 54 and introduced to the top of the second distillation column 50. The lower stream 52 is removed from the bottom of second distillation column 50 and satisfies at least one of conditions (i) to (iii). The organic phase 62 from vessel 60 is recycled to a lower portion of second distillation column 50. The organic phase 62 being rich in methyl iodide as compared to the second mixture, may be recycled to a position lower than the position for withdrawing the sidedraw 53, e.g., lower than the collector tray 55.

The portion of sidedraw 53 contains relatively more acetaldehyde in the aqueous phase 61. Aqueous phase 61, owing to its water content, may present as a suitable extracting mixture for second distillation column 50 and a portion thereof in line 63 may be recycled as the extractant 54. This recycle portion in line 63 may comprise the whole extractant 54 or may be combined with additional sources of water to comprise a portion of the extractant 54. In one embodiment, aqueous phase 61 is not used as an extracting mixture and line 63 may be have a closable valve or line 63 may be removed from the process.

Similar to the relatively high temperature of the sidedraw 53 (second mixture), the aqueous phase 61 from vessel 60 may be cooled by passing through a condenser (cooler) prior to be collected in decanter 64. Cooling water or process water may be used as the coolant. The temperature of the aqueous phase 61 may be from −5° C. to 60° C., e.g., from 0° C. to 30° C. or from 3° C. to 20° C.

In the decanter 64, there may be a residual amount of methyl iodide that is separable by a liquid-liquid separation into a residual stream 65. The residual stream 65 contains more methyl iodide than aqueous phase 61. The residual stream 65 (a heavy phase rich in methyl iodide or a lower phase) formed in the decanter 64 is recycled to the second distillation column 50 by either being combined with the organic phase 62 of vessel 60 or being added to the second distillation column 50 below the collector plate 55. In some embodiments, residual stream 65 may bypass the second distillation column 50 and is returned to reactor 10 with lower stream 52. To prevent phase issues, it is not advisable to introduce residual stream 65 back into vessel 60.

Decanter 64 also yields a liquid stream 66. The liquid stream 66 contains the targeted acetaldehyde to be removed. The mass flow ratio of the liquid stream 66 and the residual stream 65 may be from 1:500 to 1:0.5 (liquid to residual), e.g., from 1:400 to 1:1 or from 1:375 to 1:10. Despite the smaller relative stream, the liquid stream 66 contains an useful amount of acetaldehyde. The acetaldehyde mass composition in liquid stream 66 based on amount may be more than 2× (two times) the amount in residual stream 65, e.g., more than 3× or more than 4×.

Although the liquid stream 66 may be disposed of to reduce the acetaldehyde mass composition, there may be processes which seek to further retain methyl iodide and/or the extractant (water) used for the extracting mixture. Thus, the liquid stream 66, or a portion thereof, may be further subjected to separation using a third distillation column 70. In such a distilling step, third distillation column 70 yields an overhead stream 71 containing acetaldehyde in an amount from 1 to 99 wt % and methyl iodide in an amount from 0.1 to 30 wt %, and a bottoms stream 72 containing the extractant as the main component in an amount of not less than 10 wt %, and methyl iodide in an amount of not more than 1 wt % (provided that, each stream, including impurities, has a total amount of 100% by weight). A portion of the bottoms stream 72 may be used as the extractant via line 73 and returned to second distillation column 50. In other embodiment, bottoms stream 72 may be removed or discharged from the process.

The third distillation column 70 may have a column top pressure (absolute) from 100 to 500 kPa, e.g., 115 to 375 kPa and 125 to 250 kPa. To effectively separate the overhead, the third distillation column 70 at atmospheric pressure has a temperature at the column top from 10 to 90° C., e.g., from 15 to 80° C. or 20 to 60° C., and/or a column bottom temperature from 70 to 170° C., e.g., from 80 to 160° C. or from 90 to 150° C. The number of stages (plates) in the third distillation column 70 may be a sufficient number for separation, for example, from 1 to 50 plates, e.g., from 2 to 45 plates or from 3 to 30 plates. The reflux ratio (reflux:distillate) of the third distillation column 70 is from 1:20 to 20:1, e.g., from 1:15 to 15:1, or from 5:1 to 10:1.

The overhead stream 71 or a distillate thereof contains more acetaldehyde and has a lower methyl iodide mass composition than second mixture. In one embodiment, the composition of overhead stream 71 comprises a PRC (acetaldehyde) mass composition from 45 to 99 wt %, e.g., from 50 to 99 wt % or from 60 to 98 wt %, C₁-C₁₂ alkyl iodides (methyl iodide) mass composition from 0.1 to 30 wt %, e.g., from 0.5 to 25 wt %, or from 1 to 20 wt %, methyl acetate mass composition from 0.1 to 25 wt %, e.g., from 0.5 to 20 wt %, or from 0.5 to 12 wt %, acetic acid mass composition from 0 to 5 wt %, e.g., from 0 to 1.5 wt %, or from 0 to 1 wt %, water mass composition from 0 to 5 wt %, e.g., from 0 to 2.5 wt %, or from 0.01 to 2 wt %, methanol mass composition from 0 to 2.5 wt %, e.g., from 0.01 to 2.1 wt %, or from 0.05 to 2 wt %, acetal mass composition from 0 to 2.5 wt %, e.g., from 0.01 to 1.7 wt %, or from 0.05 to 1.5 wt %, and dimethyl ether mass composition from 0 to 1.2 wt %, e.g., from 0.01 to 0.8 wt %, or from 0.05 to 0.5 wt %. In one embodiment, the overhead stream 71 has a ratio (based on weight) of methyl iodide relative to acetic acid is that higher than this ratio in feed to the third distillation column 70. In addition or separately, the overhead stream 71 may have a ratio (based on weight) of methyl iodide relative to methyl acetate is that higher than this ratio in feed to the third distillation column 70.

Overhead stream 71 has a temperature at atmospheric pressure from 15 to 100° C., from 20 to 90° C. or from 35 to 75° C. A conventional condenser/cooler may be used to condense the overhead stream 71 to cool the overhead stream 71 to a temperature of not more than 60° C., e.g., not more than 45° C. or not more than 30° C.

In one embodiment, when the extractant 54 is water, the liquid in the bottoms stream 72 contains water as the main component. In addition to the main component, the bottoms stream 72 may contain methyl acetate and lower amounts of acetic acid, methanol, dimethyl ether, methyl iodide, and/or acetaldehyde. This allows a portion of bottoms stream 72 or the whole bottoms stream 72 to be used as the extractant 54 by recycling in line 73 to second distillation column 50. The bottoms stream 72 may have a water mass composition from 85 to 99.99 wt %, e.g., from 90 to 99.98 wt % or from 92 to 99 wt %. Methyl acetate may be retained in the lower part of the third distillation column 70 and is withdrawn in the lower stream 72. The mass composition of methyl acetate in the bottoms stream 72 may be from 0.1 to 15 wt %, e.g., from 0.5 to 10 wt %, or from 0.7 to 7 wt %. The other components, when present, are generally in lower individual amounts of not more than 5 wt %. In one embodiment, the bottoms stream 72 may have a mass composition of acetaldehyde of not more than 1 wt %, e.g. not more than 0.5 wt % or not more than 0.3 wt %, a mass composition of methyl iodide of not more than 1.5 wt %, e.g., not more than 1 wt %, or not more than 0.5 wt %, a mass composition of acetic acid of not more than 5 wt %, e.g., not more than 1 wt %, or not more than 0.5 wt %, a mass composition of methanol of not more than 1 wt %, e.g., not more than 0.5 wt %, or not more than 0.1 wt %, and/or a mass composition of dimethyl ether of not more than 0.1 wt %, e.g., not more than 0.01 wt %, or not more than 0.001 wt %. Bottoms stream 72 has a temperature at atmospheric pressure from 65 to 165° C., e.g., from 70 to 120° C. or from 85 to 105° C.

Although FIG. 2 shows liquid stream 66 being distilled, in other embodiments, the second mixture and/or aqueous stream 61 may be distilled in the third distillation column 70 by without passing through either vessel 60 and/or decanter 64.

Separating methyl iodide from acetaldehyde by distillation alone proves to be unable to fully recover methyl iodide, even though the methyl iodide mass composition is low in overhead stream 71. Further simple distillation may yield marginal or incremental improvements in recovering methyl iodide, thus more effective processing provides attractive benefits for supplemental processing. Extraction with or without distillation may be used as an effective process to enhance recovery of methyl iodide. In one embodiment, a second extractive distillation column may be used to enhance recovery of methyl iodide. As seen in FIG. 2, overhead stream 71 or a distillate portion thereof, is introduced to fourth distillation column 80 that operates as an extractive distillation using a water-containing extractive mixture. Fourth distillation column 80 operates in a manner to obtain an overhead stream 81 enriched in methyl iodide and an aqueous bottom stream 82 enriched in acetaldehyde as well as the extractant, being water. At least a portion, including the entire portion, of aqueous bottom stream 82 may be recycled or returned to second distillation column 50 via line 83 as the extracting mixture.

In one embodiment, the fourth distillation column 80 separates an upper stream 81 from having a ratio (based on weight) of methyl iodide relative to acetaldehyde that is greater than that of the feed in overhead (distillate) stream 71. Upper stream 81 may be taken as an overhead or a stream near the top of fourth distillation column 80. To maintain recovery, it may be useful to direct the upper stream 81, either directly or indirectly, to the reactor 10. In some embodiments, a portion of the upper stream 81 may be introduced to the second distillation column 50, preferably in a lower portion.

For extraction, it is sufficient to add the water-extracting mixture in a counter-current direction at the top of the fourth distillation column 80 via line 84. As described in U.S. Pat. No. 8,859,810, the entire contents and disclosure of which are incorporated by reference, the water-extracting mixture may comprise water, glycols, glycerol, high boiling point alcohols, including mixtures thereof. For the water extractive distillation, the water may have the same temperature as the extractant. The water may be added as a warmed or heated water having the same temperature as the extractant or as a vaporized water (or steam). In one embodiment, the water-extracting mixture 84 has a temperature that is controlled or maintained to be within the range of 0 to 60° C., e.g., 10 to 50° C. or 20 to 40° C. The weight ratio of the flow rate of the water-extracting mixture 84 relative to the flow rate of the overhead stream 71 or a distillate portion thereof [the former/the latter] may range from 1:1000 to 10:1, e.g., from 1:500 to 5:1, 1:100 to 5:1 or 1:4 to 4:1.

In fourth distillation column 80, the overhead stream 81 is cooled and/or condensed, e.g.

by passing through a condenser (indirect condenser) and a first portion of the condensate is returned or refluxed to the distillation column 80, while a second portion of the condensate is recycled to the reactor 10 in FIG. 1. Bottom stream 82 is a liquid stream and can be withdrawn in the lower portion of distillation column 80, including the bottom or near the bottom, and contains acetaldehyde and the extractant. Owing to the enriched acetaldehyde, liquid stream 82 is purged or discharged outside of the system. A portion of the liquid stream 82 may be used an extractant in either the second distillation column 50 and/or fourth distillation column 80. The overhead stream 81 has a weight ratio of methyl iodide to acetaldehyde that is larger than the methyl iodide to acetaldehyde in liquid stream 82.

The fourth distillation column 80 may have a column top pressure (absolute) from 100 to 500 kPa, e.g., 100 to 400 kPa and 105 to 350 kPa. To effectively separate the overhead, the fourth distillation column 80 at atmospheric pressure has a temperature at the column top from 10 to 90° C., e.g., from 15 to 80° C. or 20 to 60° C., and/or a column bottom temperature from 70 to 170° C., e.g., from 80 to 160° C. or from 90 to 150° C. The number of stages (plates) in the fourth distillation column 80 may be a sufficient number for separation, for example, from 1 to 50 plates, e.g., from 2 to 45 plates or from 3 to 30 plates. The reflux ratio (reflux:distillate) of the fourth distillation column 80 is from 1:20 to 20:1, e.g., from 1:15 to 15:1, or from 5:1 to 10:1.

In one embodiment, the fourth distillation column 80 may have a theoretical stage (or plate) of, for example, less than 50 plates, overhead stream 81 or a condensed portion thereof may have a methyl iodide mass composition from 20 to 80 wt %, e.g., 30 to 75 wt % or 40 to 65 wt %, PRC mass composition from 0.1 to 70 wt %, e.g., from 0.5 to 65 wt %, or from 1 to 20 wt %, methyl acetate mass composition from 0.01 to 15 wt %, e.g., from 0.05 to 10 wt %, or from 0.1 to 10 wt %, acetic acid mass composition from 0 to 5 wt %, e.g., from 0 to 3 wt %, or from 0 to 1 wt %, and water mass composition from 0 to 10 wt %, from 0 to 8 wt %, or from 0.01 to 5 wt %. The mass composition of other organics, such as dimethyl ether and/or methanol, in the overhead stream 81 may be in a minor portion, e.g., not more than 1 wt % or not more than 0.5 wt %. Also when the fourth distillation column 80 contains less than 50 plates, the bottom stream 82 may have a PRC mass composition from 1 to 90 wt %, e.g., from 5 to 80 wt %, or from 10 to 50 wt %, water mass composition from 10 to 95 wt %, from 15 to 90 wt %, or from 20 to 85 wt %, methyl iodide mass composition from 0 to 2 wt %, e.g., 0.01 to 1.5 wt % or 0.05 to 1 wt %, methyl acetate mass composition from 0.01 to 15 wt %, e.g., from 0.05 to 10 wt %, or from 0.1 to 10 wt %, acetic acid mass composition from 0 to 5 wt %, e.g., from 0 to 3 wt %, or from 0 to 1 wt %, and a mass composition of organics (dimethyl ether and/or methanol) not more than 3 wt %, e.g., not more than 1 wt % or not more than 0.5 wt %. When the bottom liquid 82 is discharged and/or purged from the process, the acetaldehyde to methyl iodide mass ratio may be from 20:1 to 2000:1, e.g., from 35:1 to 1800:1 or from 50:1 to 1000:1.

In the continuous process to produce acetic acid the process streams, both vapor or liquid streams, may contain various components that are impurities although not described in detail above. These impurities may be formed in the reactor through side reactions. To avoid such impurities it is desirable to suppress the formation of impurities or purge the impurities to prevent build up. The various process stream may contain various amounts formic acid, higher acids, and/or hydrogen iodide.

There may be various configurations of separation process shown in FIG. 2. This includes additional units that supplement or replace the third and/or fourth distillation columns. This allows liquid stream 66 from decanter 64 to bypass third distillation column 70 and is fed into the fourth distillation column 80 or may be fed to one or more extraction vessels. Thus, if necessary, acetaldehyde may be extracted with water from the liquid stream 66 by one or a plurality of water extraction vessel that are provided with a mixer and a settler or by the fourth distillation column 80. In other embodiments, it may not be necessary to use third and/or fourth distillation columns to purify liquid stream 66.

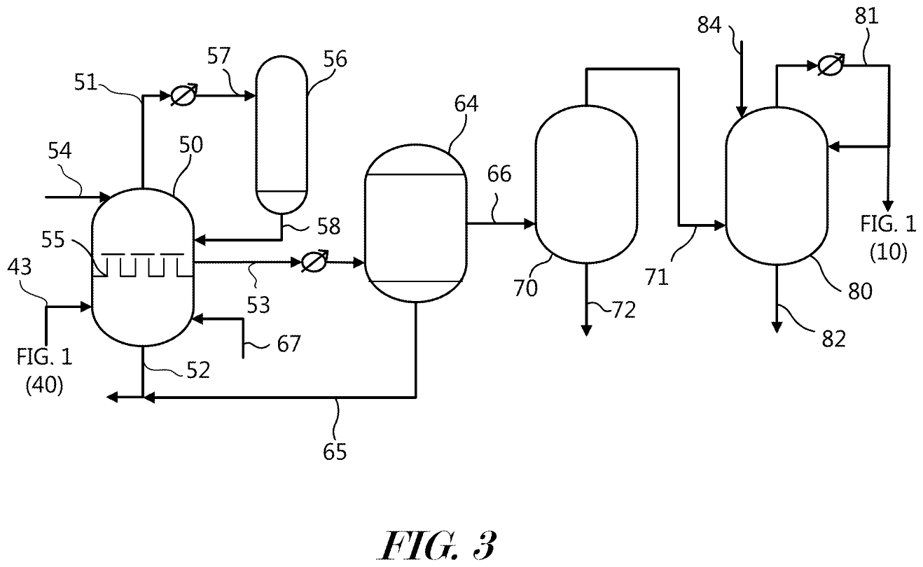

FIG. 3 represents another embodiment that provides a separation process for supplemental acetaldehyde removal process. In one embodiment, feed line 43 to the second distillation column 50 has the lower phase 42a from receiver 40 in FIG. 1. This allows feed line 43 to contain C₁-C₁₂ alkyl iodides (mainly represented by methyl iodide) in an amount from 60 to 98 wt %, e.g., from 60 to 95 wt % or from 75 to 93 wt %, PRC (acetaldehyde) in an amount of up to 5 wt %, e.g., up to 3 wt % or up to 0.5 wt %, and water in an amount up to 3 wt %, e.g., up to 1 wt % or up to 0.8 wt %. Further, feed line also contains low amounts of methanol and if the methanol needs to be adjusted feed line can be made to comprise a portion of upper phase. As described above, an extractant is added via line 54 above the collector tray 55. Any vapors at the top are collected, condensed and refluxed to the second distillation column 50.

In this embodiment, sidestream 53 is condensed or chilled, from −5° C. to 60° C., for direct feeding to decanter 64, thus skipping vessel 60 in FIG. 2, for liquid-liquid separation to obtain a residual stream 65 (containing methyl iodide) and a liquid stream 66 (containing acetaldehyde). The mass flow ratio of the liquid stream 66 and the residual stream 65 may be from 1:500 to 1:0.5 (liquid to residual), e.g., from 1:400 to 1:1 or from 1:375 to 1:10. Sidestream 53 may have a composition is suitable of phase separation and in one embodiment, the composition of the sidestream 53 may have a PRC mass composition from 0.1 to 90 wt %, e.g., from 0.2 to 65 wt % or from 0.5 to 50 wt %, C₁-C₁₂ alkyl iodides (methyl iodide) mass composition from 0.5 to 95 wt %, e.g., from 1 to 95 wt %, from 5 to 90 wt %, or from 10 to 60 wt %, methyl acetate mass composition from 0.1 to 25 wt %, e.g., from 0.5 to 20 wt %, or from 0.5 to 10 wt %, acetic acid mass composition from 0 to 10 wt %, e.g., from 0.01 to 5 wt %, or from 0.05 to 1 wt %, water mass composition from 0.1 to 20 wt %, e.g., from 0.5 to 15 wt %, or from 0.5 to 8 wt %, methanol mass composition from 0 to 2.5 wt %, e.g., from 0.01 to 2.1 wt %, or from 0.05 to 2 wt %, acetal mass composition from 0 to 2.5 wt %, e.g., from 0.01 to 1.7 wt %, or from 0.05 to 1.5 wt %, and dimethyl ether mass composition from 0 to 1.2 wt %, e.g., from 0.01 to 0.8 wt %, or from 0.05 to 0.5 wt %. The process as shown in FIG. 3 further concentrates the PRC without building up large quantities of acetals.

As shown in FIG. 3, residual stream 65 combined with the lower stream 52 from the second distillation column 50. In some embodiments, residual stream 65 may be fed to the lower portion of the second distillation column 50. Acetic acid, as a miscible solvent, was fed via a feed line 67 to the lower portion of second distillation column 50, and may in some embodiments be fed below the feed location of stream 43. Although not shown in FIG. 2, there may be a miscible solvent fed to the second distillation column 50.

Once withdrawn from decanter 64, liquid stream 66 is fed to the third distillation column 70. Despite the smaller relative stream, the liquid stream 66 contains an useful amount of acetaldehyde. The acetaldehyde mass composition in liquid stream 66 based on amount may be more than 2× the amount in residual stream 65, e.g., more than 3× or more than 4×. As described above, third distillation column 70 operates to yield an overhead stream 71 containing acetaldehyde in an amount from 1 to 99 wt % and methyl iodide in an amount from 0.1 to 30 wt %, and a bottoms stream 72 containing the extractant as the main component in an amount of not less than 10 wt %, and methyl iodide in an amount of not more than 1 wt % (provided that, each stream, including impurities, has a total amount of 100% by weight). A portion of the bottoms stream 72 may be used as the extractant via line 73 and returned to second distillation column 50. In other embodiment, bottoms stream 72 may be removed or discharged from the process.

The overhead stream 71 or a distillate thereof contain more acetaldehyde and has a lower methyl iodide mass composition than second mixture. In one embodiment, the composition of overhead stream 71 comprises a PRC (acetaldehyde) mass composition from 45 to 99 wt %, e.g., from 50 to 99 wt % or from 60 to 98 wt %, C₁-C₁₂ alkyl iodides (methyl iodide) mass composition from 0.1 to 30 wt %, e.g., from 0.5 to 25 wt %, or from 1 to 20 wt %, methyl acetate mass composition from 0.1 to 25 wt %, e.g., from 0.5 to 20 wt %, or from 0.5 to 12 wt %, acetic acid mass composition from 0 to 5 wt %, e.g., from 0 to 1.5 wt %, or from 0 to 1 wt %, water mass composition from 0 to 5 wt %, e.g., from 0 to 2.5 wt %, or from 0.01 to 2 wt %, methanol mass composition from 0 to 2.5 wt %, e.g., from 0.01 to 2.1 wt %, or from 0.05 to 2 wt %, acetal mass composition from 0 to 2.5 wt %, e.g., from 0.01 to 1.7 wt %, or from 0.05 to 1.5 wt %, and dimethyl ether mass composition from 0 to 1.2 wt %, e.g., from 0.01 to 0.8 wt %, or from 0.05 to 0.5 wt %. In one embodiment, the overhead stream 71 has a ratio (based on weight) of methyl iodide relative to acetic acid is that higher than this ratio in feed to the third distillation column 70. In addition or separately, the overhead stream 71 may have a ratio (based on weight) of methyl iodide relative to methyl acetate is that higher than this ratio in feed to the third distillation column 70. The bottoms stream 72 may have a water mass composition from 85 to 99.99 wt %, e.g., from 90 to 99.98 wt % or from 92 to 99 wt %. In one embodiment, bottoms stream 72 is removed from the process or at least a portion thereof may be returned as the extractant to the second distillation column 50.

Similar to FIG. 2, FIG. 3 processes the overhead stream 71 or a distillate portion thereof, by introducing this stream to the fourth distillation column 80 that operates as an extractive distillation using a water-containing extractive mixture. As described above, fourth distillation column 80 operates in a manner with an water-extracting mixture via line 84 to obtain an overhead stream 81 enriched in methyl iodide and an aqueous bottom stream 82 enriched in acetaldehyde as well as the extractant, being water. At least a portion, including the entire portion, of aqueous bottom stream 82 may be recycled or returned to second distillation column 50 via line 83 as the extracting mixture.

The material of each member or unit associated with the distillation system, including the columns, valves, condensers, receivers, pumps, reboilers, and internals, and various lines, each communicating to the distillation system may be made of suitable materials such as glass, metal, ceramic, or combinations thereof, and is not particularly limited to a specific one. According to the present disclosure, the material of the foregoing distillation system and various lines are a transition metal or a transition-metal-based alloy such as iron alloy, e.g., a stainless steel, nickel or nickel alloy, zirconium or zirconium alloy thereof, titanium or titanium alloy thereof, or aluminum alloy. Suitable iron-based alloys include those containing iron as a main component, e.g., a stainless steel that also comprises chromium, nickel, molybdenum and others. Suitable nickel-based alloys include those containing nickel as a main component and one or more of chromium, iron, cobalt, molybdenum, tungsten, manganese, and others, e.g., HASTELLOY™ and INCONEL™. Corrosion-resistant metals may be particularly suitable as materials for the distillation system and various lines.

The present invention will be better understood in view of the following non-limiting examples.

### EXAMPLES

### Example 1

A mixture containing a permanganate reducing compound (PRC), one or more C₁-C₁₂ alkyl iodides and water was distilled in an extractive distillation column using a semi-empirical simulator. The PRC for this example is acetaldehyde and the primarily C₁-C₁₂ alkyl iodide is methyl iodide. This mixture also contained methanol as shown in Table 3.

The extractive distillation column operated with a top temperature of 37° C. and a bottom temperature of 44° C. The pressure of the extractive distillation column was 0 psig. The extractive distillation had 45 plates. The extractant was a water-containing stream and the extractant was fed to the top plate. The extractant did not contain methanol and no other sources of methanol were fed to the extractive distillation column other than the first mixture. The temperature of the extractant was 20° C.

As a result of extractive distillation a second mixture was obtained from the side of the extractive distillation column. The second mixture was withdrawn from a collector tray positioned above the feed point of the first mixture. Also a lower stream was removed from the bottom of the extractive distillation column, which is below the feed point of the first mixture. The compositions for the second mixture and lower stream are shown in Table 3 as well.

TABLE 3
Extractive Distillation Column (wt %)

| | First | Second | Lower |
|---|---|---|---|
| | Mixture | Mixture | Stream |
| Methyl Iodide | 82.5 | 78.27 | 85.88 |
| Methyl Acetate | 14.8 | 6.12 | 16.28 |
| Acetic Acid | 1.83 | 0.02 | 2.2 |
| Acetaldehyde | 0.196 | 1.10 | 0.0048 |
| Methanol | 0.1128 | 1.05 | 0.036 |
| Water | 0.701 | 13.28 | 0.488 |
| 1,1-dimethoxyethane | 0 | 0 | 0.0172 |

The difference based on weight of methyl iodide and water in the first mixture was 81.8 wt % (82.5 wt %−0.701 wt %) and the methanol mass composition (0.1128 wt %) was maintained below this difference. As shown by Table 3, the second mixture was enriched in terms of acetaldehyde over the first mixture. This indicates the effectiveness in separating the acetaldehyde using the extractive distillation column. The total acetal mass composition in the lower stream satisfied condition (i). The combined mass composition of methanol and acetaldehyde (0.138 wt %) is more than the 1,1-dimethoxyethane mass composition and condition (ii) is satisfied. Further, as compared to the total methanol mass composition in the first mixture being 1128 ppm, the 1,1-dimethoxyethane mass composition is lower and condition (iii) is satisfied.

### Example 2

Using the extractive distillation column of Example 1, the following mixture shown in Table 4 was fed. The second mixture was obtained from the side and a lower stream obtained from the bottom as was done in Example 1. Table 4 reports the results.

TABLE 4
Extractive Distillation Column (wt %)

| | First | Second | Lower |
|---|---|---|---|
| | Mixture | Mixture | Stream |
| Methyl Iodide | 82.5 | 92.92 | 78.2 |
| Methyl Acetate | 14.78 | 3.85 | 19.17 |
| Acetic Acid | 1.83 | 0 | 2.51 |
| Acetaldehyde | 0.196 | 0.59 | 0.02 |
| Methanol | 0.0033 | 0.02 | 0 |
| Water | 0.70 | 2.6 | 0.1 |
| 1,1-dimethoxy ethane | 0 | 0 | 0 |

The difference, based on mass, of methyl iodide and water in the first mixture was 81.8 wt % and the methanol mass composition was maintained well this difference. As shown by Table 4, the second mixture was enriched in terms of acetaldehyde over the first mixture. This indicates the effectiveness in separating the acetaldehyde using the extractive distillation column. The total acetal mass composition in the lower stream satisfied condition (i). The combined mass composition of methanol and acetaldehyde (0.02 wt %) is more than the 1,1-dimethoxyethane mass composition and condition (ii) is satisfied. Further, as compared to the total methanol mass composition in the first mixture being 33 ppm, the 1,1-dimethoxyethane mass composition is lower and condition (iii) is satisfied.

### Example 3

Using the extractive distillation column of Example 1, comparative mixtures shown in Table 5 was fed. The second mixture was obtained from the side and a lower stream obtained from the bottom as was done in Example 1. Table 5 reports the results.

TABLE 5
Extractive Distillation Column (wt %)

| | First | Second | Lower |
|---|---|---|---|
| | Mixture | Mixture | Stream |
| Methyl Iodide | 80.04 | 93.40 | 76.37 |
| Methyl Acetate | 14.77 | 2.93 | 19.52 |
| Acetic Acid | 1.83 | 0 | 2.98 |
| Acetaldehyde | 0.196 | 0.18 | 0.02 |
| Methanol | 2.52 | 1.65 | 0.81 |
| Water | 0.7 | 1.69 | 0.18 |
| 1,1-dimethoxy ethane | 0 | 0.146 | 0.11 |

Despite the higher amounts of methanol in the first mixture, the lower stream was able to satisfy conditions (ii) and (iii).

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

### Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a process for separating at least one permanganate reducing compound (PRC) from a first mixture comprising the at least one PRC, one or more C1-C12 alkyl iodides and water, the process comprising the steps of: feeding the first mixture to a first location of a distillation column, wherein the first mixture has a methanol mass composition that is less than the difference based on mass between the one or more C1-C12 alkyl iodides and water; distilling the first mixture into a second mixture and a lower stream, the second mixture being at least one stream selected from the group consisting of an overhead stream and a sidedraw stream being withdrawn higher than that of the first location; and withdrawing the lower stream from a second location lower than the first location, and wherein the lower stream satisfies at least one of the following conditions (i) to (iii): a total acetal mass composition in the lower stream is not more than 0.02 wt %; a total acetal mass composition in the lower stream is less than the combined mass composition of methanol and acetaldehyde in the lower stream; and a total acetal mass composition in the lower stream is less than the methanol mass composition in the first mixture; wherein the first mixture is separated into the second mixture and lower stream without the supply of additional methanol; and wherein the second mixture is enriched in the at least one PRC.

Embodiment 2 is the process of embodiment(s) 1, wherein the lower stream satisfies conditions (i) and (ii).

Embodiment 3 is the process of embodiment(s) 1-2, wherein the lower stream satisfies conditions (i) and (iii).

Embodiment 4 is the process of embodiment(s) 1-3, wherein the total acetal mass composition for condition (i) is from 0.0001 wt % to 0.02 wt %.

Embodiment 5 is the process of embodiment(s) 1-4, wherein the combined mass composition of methanol and acetaldehyde for condition (ii) is not more than 1.9 wt %.

Embodiment 6 is the process of embodiment(s) 1-5, wherein the methanol mass composition for condition (iii) is not more than 2 wt %.

Embodiment 7 is the process of embodiment(s) 1-6, wherein the first mixture comprises the one or more C1-C12 alkyl iodides in an amount from 2.5 wt % to 90 wt %, the at least one PRC in an amount from 0.05 to 50 wt %, and water in an amount from 0.5 wt % to 90 wt %.

Embodiment 8 is the process of embodiment(s) 1-7, wherein the methanol mass composition in the first mixture is less than or equal to 2 wt %.

Embodiment 9 is the process of embodiment(s) 1-8, wherein the first mixture is distilled using an extractant comprising not less than 80 wt % water.

Embodiment 10 is the process of embodiment(s) 9, wherein the flow rate ratio (on a weight basis) of the extractant to the first mixture is from 0.0001/100 to 100/100.

Embodiment 11 is the process of embodiment(s) 1-10, wherein the second mixture comprises the one or more C1-C12 alkyl iodides in an amount from 0.5 to 95 wt %, the at least one PRC in an amount from 0.1 to 90 wt %, and water in an amount from 0.1 to 20 wt %.

Embodiment 12 is the process of embodiment(s) 1-11, further comprising biphasically separating the second mixture into an aqueous phase and an organic phase.

Embodiment 13 is an acetic acid production process, the process comprising the following steps for controlling the formation of acetals: (a) allowing methanol to continuously react with carbon monoxide in the presence of a reactive mixture comprising a metal catalyst, an ionic metal iodide, and methyl iodide, acetic acid, methyl acetate, and water; (b) evaporating the reaction mixture with or without heating to yield a vapor crude product and a catalyst recycle stream; (c) distilling at least a portion of the vapor crude product to form an overhead and a side stream and condensing the overhead in one or more condensers and collecting the condensate(s) in a receiver into an upper phase and a lower phase; and (d) separating at least a portion of the lower phase to form a second stream comprising acetaldehyde and a lower stream, wherein the lower stream satisfies at least one of the following conditions (i) to (iii): a total acetal mass composition in the lower stream is not more than 0.02 wt %; a total acetal mass composition in the lower stream is less than the combined mass composition of methanol and acetaldehyde in the lower stream; or a total acetal mass composition in the lower stream is less than the methanol mass composition in the lower phase; wherein the lower phase is separated into the second stream and lower stream without the supply of additional methanol; and wherein the second stream is enriched in at least one PRC.

Embodiment 14 is the process of embodiment(s) 13, wherein the lower phase comprises one or more C1-C12 alkyl iodides in an amount from 60 to 98 wt %, the at least one PRC in an amount of up to 5 wt %, water in an amount of up to 3 wt %, and methanol in an amount of up to 5 wt %.

Embodiment 15 is the process of embodiment(s) 13-14, wherein step (d) further comprises separating at least a portion of the upper phase, wherein step (d) separates a mixture having a weight ratio of the lower phase to the upper phase from 100:0 to 10:90.

Embodiment 16 is the process of embodiment(s) 13-15, wherein the lower stream is returned to step (a) or step (d).

Embodiment 17 is the process of embodiment(s) 13-16, wherein the at least a portion of the lower phase is distilled using an extractant comprising not less than 80 wt % of water.

Embodiment 18 is the process of embodiment(s) 13-17, wherein the total acetal mass composition for condition (i) is from 0.0001 wt % to 0.02 wt %.

Embodiment 19 is the process of embodiment(s) 13-18, wherein the combined mass composition of methanol and acetaldehyde for condition (ii) is not more than 1.9 wt %.

Embodiment 20 is the process of embodiment(s) 13-19, wherein the methanol mass composition for condition (iii) is not more than 2 wt %.

1. A process for separating at least one permanganate reducing compound (PRC) from a first mixture comprising the at least one PRC, one or more C₁-C₁₂ alkyl iodides and water, the process comprising the steps of:
feeding the first mixture to a first location of a distillation column, wherein the first mixture has a methanol mass composition that is less than the difference based on mass between the one or more C₁-C₁₂ alkyl iodides and water;
distilling the first mixture into a second mixture and a lower stream, the second mixture being at least one stream selected from the group consisting of an overhead stream and a sidedraw stream being withdrawn higher than that of the first location; and
withdrawing the lower stream from a second location lower than the first location, and wherein the lower stream satisfies at least one of the following conditions (i) to (iii):
a total acetal mass composition in the lower stream is not more than 0.02 wt %;
(ii) a total acetal mass composition in the lower stream is less than a combined mass composition of methanol and acetaldehyde in the lower stream; and
(iii) a total acetal mass composition in the lower stream is less than the methanol mass composition in the first mixture;
wherein the first mixture is separated into the second mixture and lower stream without the supply of additional methanol; and
wherein the second mixture is enriched in the at least one PRC.

2. The process of claim 1, wherein the lower stream satisfies conditions (i) and (ii).

3. The process of claim 1, wherein the lower stream satisfies conditions (i) and (iii).

4. The process of claim 1, wherein the total acetal mass composition for condition (i) is from 0.0001 wt % to 0.02 wt %.

5. The process of claim 1, wherein the combined mass composition of methanol and acetaldehyde for condition (ii) is not more than 1.9 wt %.

6. The process of claim 1, wherein the methanol mass composition for condition (iii) is not more than 2 wt %.

7. The process of claim 1, wherein the first mixture comprises the one or more C₁-C₁₂ alkyl iodides in an amount from 2.5 wt % to 90 wt %, the at least one PRC in an amount from 0.05 to 50 wt %, and water in an amount from 0.5 wt % to 90 wt %.

8. The process of claim 1, wherein the methanol mass composition in the first mixture is less than or equal to 2 wt %.

9. The process of claim 1, wherein the first mixture is distilled using an extractant comprising not less than 80 wt % water.

10. The process of claim 9, wherein a ratio of the flow rate (on a weight basis) of the extractant to the first mixture is from 0.0001/100 to 100/100.

11. The process of claim 1, wherein the second mixture comprises the one or more C₁-C₁₂ alkyl iodides in an amount from 0.5 to 95 wt %, the at least one PRC in an amount from 0.1 to 90 wt %, and water in an amount from 0.1 to 20 wt %.

12. The process of claim 1, further comprising biphasically separating the second mixture into an aqueous phase and an organic phase.

13. An acetic acid production process, the process comprising the following steps for controlling the formation of acetals:
(a) allowing methanol to continuously react with carbon monoxide in the presence of a reaction mixture comprising a metal catalyst, an ionic metal iodide, and methyl iodide, acetic acid, methyl acetate, and water;
(b) evaporating the reaction mixture with or without heating to yield a vapor crude product and a catalyst recycle stream;
(c) distilling at least a portion of the vapor crude product to form an overhead and a side stream and condensing the overhead in one or more condensers and collecting the condensate(s) in a receiver into an upper phase and a lower phase; and
(d) separating at least a portion of the lower phase to form a second stream comprising acetaldehyde and a lower stream, wherein the lower stream satisfies at least one of the following conditions (i) to (iii):
a total acetal mass composition in the lower stream is not more than 0.02 wt %;
(ii) a total acetal mass composition in the lower stream is less than a combined mass composition of methanol and acetaldehyde in the lower stream; or
(iii) a total acetal mass composition in the lower stream is less than the methanol mass composition in the lower phase;
wherein the lower phase is separated into the second stream and lower stream without the supply of additional methanol; and
wherein the second stream is enriched in at least one PRC.

14. The process of claim 13, wherein the lower phase comprises one or more C₁-C₁₂ alkyl iodides in an amount from 60 to 98 wt %, the at least one PRC in an amount of up to 5 wt %, water in an amount of up to 3 wt %, and methanol in an amount of up to 5 wt %.

15. The process of claim 13, wherein step (d) further comprises separating at least a portion of the upper phase, wherein step (d) separates a mixture having a weight ratio of the lower phase to the upper phase from 100:0 to 10:90.

16. The process of claim 13, wherein the lower stream is returned to step (a) or step (d).

17. The process of claim 13, wherein the at least a portion of the lower phase is distilled using an extractant comprising not less than 80 wt % of water.

18. The process of claim 13, wherein the total acetal mass composition for condition (i) is from 0.0001 wt % to 0.02 wt %.

19. The process of claim 13, wherein the combined mass composition of methanol and acetaldehyde for condition (ii) is not more than 1.9 wt %.

20. The process of claim 13, wherein the methanol mass composition for condition (iii) is not more than 2 wt %.
